(19) United States
(12) Patent Application Publication
(10) Pub. No.: US 2010/0069930 A1
(43) Pub. Date: Mar. 18, 2010

(54) Method and apparatus for minimally invasive delivery, tensioned deployment and fixation of secondary material prosthetic devices in patient body tissue, including hernia repair within the patient's herniation site

(75) Inventors: Mitchell Roslin, Armonk, NY (US); Paresh C. Shah, Riverside, CT (US); Oleg Shikhman, Trumbull, CT (US); Danial P. Ferreira, Milford, CT (US); Jeffrey P. Radziunas, Wallingford, CT (US); Christopher A. Battles, Seymour, CT (US)

Correspondence Address: MICHAELSON & ASSOCIATES, P.O. BOX 8489, RED BANK, NJ, 07701-8489, US

(73) Assignee: VentralFix, Inc., Armonk, NY (US)

(21) Appl. No.: 12/586,021

(22) Filed: Sep. 16, 2009

Related U.S. Application Data
(60) Provisional application No. 61/192,208, filed on Sep. 16, 2008.
(60) Provisional application No. 61/214,316, filed on Apr. 21, 2009.

(51) Int. Cl. A61B 17/08

(52) U.S. Cl. 606/151

(57) ABSTRACT

Apparatus and methods enable insertion and tensioned deployment of a secondary material prosthetic device into a body cavity or other tissue of a patient, such as for example hernia repair mesh into the abdominopelvic cavity of a patient through the hernia site. The present invention establishes fixation sites for the prosthetic device and tensions it against the body tissue. It may also be used implant fixation devices within the body tissue so that the prosthetic device is tensioned into firm abutting contact with the body tissue. Instrument deployment and fixation struts may be advanced in retrograde fashion in order to reduce needed deployment volume within the patient's body cavity. The prosthetic device advantageously may be flexibly coupled to the instrument via fixation devices such as sutures, so as to increase orientation flexibility.

### CLAIM TO PRIORITY

This application claims priority under 35 U.S.C. §119 (e) to the following U.S. provisional applications: Ser. No. 61/192,208 filed on Sep. 16, 2008 and Ser. No. 61/214,316 filed on Apr. 21, 2009, each of which is hereby incorporated by reference in its entirety.

### BACKGROUND OF THE DISCLOSURE

1. Field of the Invention

The invention relates to methods and apparatus minimally invasive delivery, tensioned deployment and fixation of secondary material, including prosthetic devices, in a selected implantation site defined by a patient's body tissue. The tissue may include tissue within or defining a body cavity. An exemplary, non-limiting application of this invention is for repair of a herniation in a wall of the abdominopelvic cavity of a patient.

2. Description of the Prior Art

A hernia is a weakness or hole within a patient's abdominopelvic wall that may allow internal organ tissue, such as intestines or bowel, to ex-filtrate the abdominal cavity through the herniation site and potentially become entrapped within the herniation site. Common types of hernias include umbilical, inguinal and ventral hernias.

Known treatment for hernias entails surgical repair of the herniation site by closing the hole or weakness in the abdominopelvic cavity. Today a common surgical repair technique is to introduce a mesh within the abdominopelvic cavity over the herniation site, so as to add a reinforcing “patch” to the wall. Hernia repair with mesh is analogous to repairing an automotive tire puncture with a patch placed over the puncture from the tire interior. The hernia repair mesh is spread over the herniation site and affixed to the parietal peritoneum layer of the patient's internal abdominal wall in abutting relationship. The mesh prevents organ tissue ex-filtration through the abdominal wall herniation site.

In one known “open surgery” hernia repair method, the mesh is introduced invasively into the abdominal cavity through surgical incision and dissection. The surgeon forms an incision on the order of four inches (ten centimeters) or greater into the patient's abdomen that is sufficiently large to introduce the mesh into the abdominal cavity and allow passage of surgical instruments therein that are necessary for insertion and stretching of the mesh over the hernia site, and affixation of the mesh to the patient's internal abdominal wall.

In more recent years a second hernia repair method has been developed though use of relatively less invasive laparoscopic surgical techniques. Laparoscopic techniques allow smaller incisions than traditional open surgical techniques. Multiple cannulae are inserted laterally through the patient's abdomen via trocars for access to the abdominal cavity. The repair mesh is rolled or otherwise collapsed and inserted into the abdominal cavity through a cannula. Laparoscopic instruments are inserted through one or more other cannulae so that the mesh may be unfurled, stretched, and affixed to the patient's abdominal wall over the hernia site. Examples of laparoscopic implantation methods and instruments for hernia repair and other medical procedures are referenced in U.S. Patents and Patent Publications Nos. 5,383,477; 5,405,360; 2007/0185506; 2008/0195121; and 2009/0125041. Generally, such instruments are inserted into the patient's body cavity, and thereafter arms or struts are extended to unfurl structurally reinforced or flaccid sheet repair mesh. The arms are abutted against the tissue surface to be repaired by a pushing motion in the same direction as the arm deployment. In other words, the arms extend generally in a forward plane from the instrument's distal tip. Pushing the instrument distal tip laterally across a relatively confined body cavity space between the cavity walls and viscera and thereafter against resilient, pliable body tissue does not always establish tensioned, taut abutting contact between the prosthetic device and the tissue. Confined body cavity space inhibits deployment of instrument arms and proper unfurling/tensioning of mesh material. Once the mesh is oriented in the desired fixation site location, it is affixed to the tissue with additional instruments or fixation instruments attached to the insertion instrument.

However, both known open surgery and laparoscopic surgery hernia repair techniques require lateral incisions into the patient so that the repair mesh can be stretched over the herniation site prior to affixation to the patient's interior abdominal wall. It is difficult to tension a sheet of planar mesh across the patient's generally cylindrical, concave inner abdominopelvic wall via laterally oriented access points. Loose or flaccid mesh may not provide sufficient structural integrity for the hernia repair and may necessitate future remedial repair. Mesh that is not properly tensioned over and affixed to the parietal peritoneum layer of the abdominal wall may not have sufficient structural integrity to inhibit ex-filtration of internal organ tissue through the existing herniation site or under the marginal edges of the mesh patch.

U.S. Patents and Patent Publications Nos. 5,397,331; 6,214,020; 6,966,916; 2002/0103494; 2005/0256532; 2007/0260179; 2008/0188874; and 2008/0306497 reference implantation of planar prosthetic repair devices for hernia repair or other medical procedures that repair voids in patient tissue directly through the void and/or by lateral placement over the void. In some of the patents there is reference to repair prosthesis devices that include structural reinforcements or tacking barbs to enhance abutment of the device and the underlying patient tissue. Such devices generally incorporate umbrella like structures that are introduced into a patient's body cavity in a folded state. Once the structure is inserted into the patient's body cavity the umbrella structure is opened and pulled against the body cavity over the tissue void. Depending on the design, the umbrella supporting rib structure is left in situ or removed from the patient. Compared to laterally introduced laparoscopic instruments, such direct insertion instruments require less maneuvering to the tissue repair/implantation site. However, the umbrella-like ribs require relatively large free volumetric space between the viscera and body cavity walls so that the umbrella may deploy. Generally the instrumentation referred to in the above-identified US patents deploy the instrument ribs in the same direction that they were inserted in the patient's body. Hence, the ribs need considerable space to complete their motion from the pre-deployed to deployed states.

Conversely, other patent documents reference that void repairs can be accomplished by suturing devices alone without any secondary prosthetic repair material, including U.S. Patents and Patent Publications Nos. 4,621,640; 4,935,027; 5,741,277; 2004/0068273; 2007/0270890; 2008/0294001; and 2009/0125039. Such references do not address solutions for surgeons who want or need to implant a prosthesis device as part of a medical procedure.

Thus, a need exists in the art for a minimally invasive hernia repair procedure and apparatus that minimizes the need for lateral incisions in a patient, and that preferably allows direct repair at the herniation site, with tensioned deployment and fixation of the repair prosthetic device to the patient's tissue at the implantation site.

A need also exists generally in the art for minimally invasive prosthesis implantation procedures and apparatus that minimize the need for multiple lateral incisions in a patient, and that preferably allow direct prosthesis fixation at any selected implantation site in patient tissue. There is a great need for such implantation procedures and apparatus that are directed to body cavity implantation, including the abdominopelvic cavity.

### SUMMARY OF THE INVENTION

The present invention apparatus and methods enable insertion, selective orientation and tensioned deployment of a secondary material, including prosthetic devices, at a selected implantation site within a patient's body tissue, that may include generally tissue within or forming a body cavity. The implantation procedures and apparatus of the present invention are suitable to reinforce tissue closure sites or to repair tissue defects, but they are not limited to repair of body cavity structural tissue: they can be applied to other body and organ tissue that are accessible within body cavities.

An exemplary secondary material is hernia repair mesh for insertion into the abdominopelvic cavity of a patient through the hernia site. With the present invention apparatus and methods, the secondary material, such as hernia repair mesh, is unfurled and circumferentially tensioned into a taut planar sheet that is selectively oriented relative to the target tissue site, such as three-dimensional, concave abdominopelvic cavity inner peritoneal layer, for selective affixation thereto. The present invention methods and apparatus enable snug and tight abutment of the secondary material at fixation points with selected tissue within the patient's body cavity, such as the abdominal cavity peritoneal layer and thereafter secure affixation of the said secondary material.

The present invention enables minimally invasive, selective orientation and tensioned deployment of a secondary material into a patient's body cavity. For example, a hernia can be repaired directly at the herniation site through a relatively small incision of approximately 0.2 inch (5 millimeters) to 2 inches (50 mm). Some embodiments of the apparatus instrumentation of the present invention and the inventive methods for their use may circumferentially pretension the secondary material, such as hernia repair mesh, into a relatively taught planar sheet that may be oriented and abutted against the target tissue, such as generally concave, three-dimensional abdominopelvic interior wall of a patient. The tensioned, abutting alignment of the secondary material increases likelihood of successful structurally sound marginal affixation of said material to the patient's target tissue. In the example of hernia repair, tensioned mesh provides additional structural integrity to the repaired herniation site, thereby reducing likelihood of future ex-filtration of the patient's abdominal organ tissue through the repaired site.

The present invention, among other things, is directed to method for implanting a prosthetic device in a patient's tissue. The inventive method comprises introducing in proximity to an implantation site in a patient's tissue a prosthetic device having a plurality of generally linear fixation devices coupled to the prosthetic device. Next the surgeon advances a fixation instrument having a distal tip along an advancement path in proximity to the implantation site. Thereafter the surgeon establishes a plurality of fixation sites in the patient's tissue for implantation of the prosthetic device by deploying from the fixation instrument distal tip at least one leg corresponding to each fixation site. The respective leg during deployment is abutted against the patient tissue. The surgeon orients the prosthetic device into tensioned abutting contact with the patient tissue at each fixation site by maneuvering the generally linear fixation devices coupled thereto in cooperation with the corresponding leg.

The present invention is also directed towards a method for implanting a prosthetic device in a patient's tissue, comprising introducing in proximity to an implantation site in a patient's tissue a prosthetic device having a plurality of fixation devices coupled thereto. Thereafter a fixation instrument having a distal tip is advanced along an advancement path in proximity to the implantation site. A plurality of fixation sites is established in the patient's tissue for implantation of the prosthetic device by deploying from the fixation instrument distal tip at least one leg corresponding to each fixation site, the respective leg deployment translating a retrograde path relative to the advancement path, and abutting the leg against the patient tissue. The prosthetic device is oriented into tensioned abutting contact with the patient tissue at each fixation site by maneuvering the fixation devices coupled thereto in cooperation with the corresponding leg.

The present invention is also directed to a method for operating a prosthetic implantation apparatus for implanting a prosthetic device in a patient's tissue. The subject apparatus has a fixation instrument having a distal tip for advancement into patient tissue and a plurality of legs deployable from the distal tip along a respective deployment path. The legs are capable of being coupled to generally linear fixation devices that are coupled to the prosthetic device. The present invention method comprises introducing in proximity to an implantation site in a patient's tissue a prosthetic device having a plurality of generally linear fixation devices coupled thereto. Next the fixation instrument distal tip is advanced into the patient's tissue along an advancement path in proximity to an implantation site. In this method a plurality of fixation sites are established in the patient's tissue for implantation of the prosthetic device. The fixation sites are established by deploying from the distal tip at least one leg corresponding to each fixation site, and abutting the leg against the patient tissue. The leg is coupled to a respective prosthetic device linear fixation device if not so previously coupled. After the abutting step the prosthetic device is oriented into tensioned abutting contact with the patient tissue at each fixation site by maneuvering the generally linear fixation devices coupled thereto in cooperation with the corresponding leg.

Another aspect of the present invention is directed to a method for operating a prosthetic implantation apparatus for implanting a prosthetic device in a patient's tissue, the apparatus having a fixation instrument having a distal tip for advancement into patient tissue and a plurality of legs deployable from the distal tip along a respective deployment path retrograde the intended advancement path. The legs are capable of being coupled to fixation devices coupled to the prosthetic device. The method of this aspect of the invention comprises introducing in proximity to an implantation site in a patient's tissue a prosthetic device having a plurality of fixation devices coupled thereto. The fixation instrument distal tip is advanced into the patient's tissue along an advancement path in proximity to an implantation site. A plurality of fixation sites is established in the patient's tissue for implantation of the prosthetic device by deploying from the distal tip at least one leg corresponding to each fixation site along a retrograde deployment path relative to the advancement path, and abutting the leg against the patient tissue. Thereafter the prosthetic device is oriented into tensioned abutting contact with the patient tissue at each fixation site by maneuvering the fixation devices coupled thereto in cooperation with the corresponding leg.

Another aspect of the present invention relates to an implantation system for implanting a prosthetic device in a patient's tissue at an implantation site. The system comprises a prosthetic device having a plurality of generally linear fixation devices coupled thereto that cooperates with a fixation instrument having a distal tip for advancement into patient tissue along an advancement path terminating proximal the implantation site. The fixation instrument has a plurality of legs deployable from the distal tip along respective deployment. Furthermore, the legs have tips for abutment against patient tissue at a fixation site established thereby. The legs are coupled to the prosthetic device generally linear fixation devices (for example sutures)during their deployment, for orienting the prosthetic device into tensioned abutting contact with the patient tissue at each fixation site by maneuvering the generally linear fixation devices in cooperation with the corresponding leg.

The present invention also relates to an implantation system for implanting a prosthetic device in a patient's tissue at an implantation site. The system comprises a prosthetic device having a plurality of fixation devices coupled thereto; and a fixation instrument having: a distal tip for advancement into patient tissue along an advancement path terminating proximal the implantation site. The system has a plurality of legs deployable from the distal tip along respective deployment paths that are retrograde the intended advancement path. The legs have tips for abutment against patient tissue at a fixation site established thereby. The legs, during their deployment, are coupled to their corresponding prosthetic device fixation devices, for orienting the prosthetic device into tensioned abutting contact with the patient tissue at each fixation site by maneuvering the fixation devices, in cooperation with the corresponding leg.

### BRIEF DESCRIPTION OF THE DRAWINGS

The teachings, of the present invention can be readily understood by considering the following detailed description in conjunction with the accompanying drawings, in which:

FIGS. 1A-5 show a telescoping tube instrumentation embodiment of the present invention;

FIGS. 6-15 show an L-shaped device body embodiment of the present invention, and alternatives for deploying the hernia repair mesh;

FIGS. 16-17 show an indexing device body embodiment of the present invention;

FIGS. 18-20 show a lever arm mesh deployment apparatus;

FIGS. 21-22 show a foldable L-shaped arm mesh deployment apparatus;

FIGS. 23-24 show a precurved memory metal tube, such as NITINOL®, mesh deployment apparatus;

FIGS. 25-27 show a mesh primary fixation device for anchoring the mesh to the patient's abdominal wall, utilizing a barbed suture;

FIGS. 28-30 show a mesh primary fixation device for anchoring the mesh to the patient's abdominal wall, utilizing a suture and pledget;

FIGS. 31-33 show a mesh primary fixation device for anchoring the mesh to the patient's abdominal wall, utilizing a suture and cleat;

FIGS. 34-36A show a mesh primary fixation device for anchoring the mesh to the patient's abdominal wall, utilizing an expanding anchor and delivery instrument;

FIGS. 37-39A show a mesh primary fixation device for anchoring the mesh to the patient's abdominal wall, utilizing an expanding braid;

FIGS. 39B-39H show a mesh primary fixation device for anchoring the mesh to the patient's abdominal wall, utilizing an inwardly-curving suture implantation path;

FIGS. 40-45 show a mesh secondary fixation device for anchoring the mesh to the patient's abdominal wall, utilizing barbed sutures with backstops. The secondary fixation supplements primary fixation at the discretion of the physician who is performing the procedure.

FIGS. 46-49A show umbrella embodiments of the present invention primary fixation device;

FIGS. 50-62B show an integrated instrumentation system embodiment of the present invention;

FIG. 63 shows schematically finished implantation of a generic prosthetic device in a patient's tissue after utilizing the methods and apparatus of the present invention;

FIG. 64 shows schematically a fixation device of the present invention oriented outside the patient's body; and

FIG. 65 shows schematically a fixation device of the present invention introduced into a patient's body cavity with laparoscopic instrumentation.

To facilitate understanding, identical reference numerals have been used, where possible, to designate identical elements that are common to the figures. Generally reference numerals 50-59 are reserved for patient anatomy designation; 60-99 for general instrumentation embodiment designations; 100-199 for insertion instrumentation apparatus designation; 200-299 for prosthetic device, including hernia repair mesh designation; 300-399 for prosthetic device, including repair mesh, unfurling, deployment and tensioning instrumentation apparatus; 400-499 for prosthetic device, including repair mesh, affixation instrumentation apparatus and 500-599 for fixation fastener devices.

### DETAILED DESCRIPTION

After considering the following description, those skilled in the art will clearly realize that the teachings of the present invention can be readily utilized in abdominopelvic hernia repairs and for implantation of other prosthetic devices within patient tissue. Implantation may be made in patient body cavities, including the abdominopelvic cavity. The implantation tissue may include tissue defining the cavity or defining organs within the cavity. For brevity herein, exemplary ventral hernia repairs will be described in connection with abdominal cavity hernias, such as umbilical or inguinal hernias.

The apparatus, prosthetic implantation and hernia repair procedures discussed herein are generally suggestive and are not intended to replace the skilled professional judgment of a licensed physician, who alone can determine their treatment suitability for any individual patient. The term “patient” may be a human being or domestic animal model used to test the efficacy of a medical device before regulatory authorities or other living creature. A “patient” includes simulated living creatures, examples of which including cadavers, synthetic physical models and computer simulated virtual models.

The apparatus and methods of the present invention enable deployment of hernia repair surgical mesh or other prosthetic devices directly at the patient's implantation site, tensioning, including circumferential tensioning of the prosthesis to assure tight abutment against the patient's tissue at the implantation site, and secure affixation to the patient's tissue.

### Repair Instrumentation

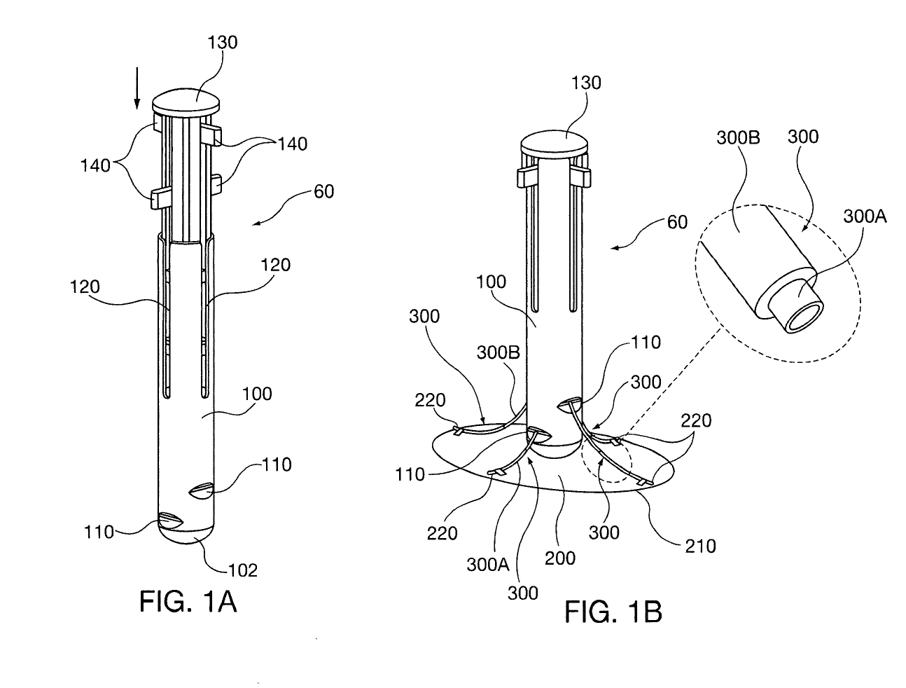

FIGS. 1A and 1B show a first embodiment of the repair instrument 60 of the present invention. The instrument 60 has an outer tubular device body 100, preferably with a distal rounded end 102 for insertion into an incision formed at the hernia site, to be discussed below in connection with operational descriptions for use of the instrument. Near the distal end 102 the device body 100 defines two opposing pairs of ports 110 in its outer periphery, oriented radially every 90 degrees. The ports might be staggered axially, for example to accommodate different shapes and dimensions of mesh. The device body 100 also defines four needle advancement channels 120 that are in axial peripheral alignment with a corresponding port 110. It is envisioned that the device body 100 and other delivery cannulae described herein preferably will have an outer diameter of between 0.5 inch (approximately 12 mm) and 2 inches (approximately 50 mm).

A plunger 130 is slidably received within the device body 100. The plunger 130 has four reciprocating needle sliders 140 that are oriented for receipt within the needle advancement channels 120. When the plunger is in its fully retracted position shown in FIG. 1A, the exterior surface of the device body 100 is generally flush. Depression of the plunger 130 to its fully inserted position shown in FIG. 1B simultaneously extends four device body legs 300, each through a separate corresponding port 110. The device body legs 300 are constructed of hollow telescoping distal 300A and proximal 300B tubular struts having the same radius of curvature to avoid binding during extension or retraction. The telescoping struts 300A, 300B have oval cross sections to inhibit torsional rotation. Any other matched non-circular cross section may be substituted for the oval cross section.

The device body legs 300, when extended, deploy the hernia repair mesh 200 in a circumferentially taut extended planar sheet generally normal to the axial dimension of the repair instrument 60. For simplicity of the drawings, the mesh 200 is not shown in FIG. 1A. As will be described in greater detail herein, when the plunger 130 is fully retracted as shown in FIG. 1A, the mesh 200 is wrapped around the lower periphery of the device body 100 or stored within the device body. Device body legs 300 are coupled to the mesh 200 by straps or pockets 220 that are affixed to or formed within the mesh that capture distal tips of the legs. Alternatively, the device body legs may be attached to the mesh 200 by a circumferential flange formed by the mesh or an auxiliary hoop affixed to the mesh circumference. An optional resilient hoop (not shown) could provide additional circumferential tension to keep the repair mesh 200 taut against the patient's abdominopelvic wall.

Another suitable way to secure the mesh 200 to the device body legs 300 of the repair instrument 60, or other embodiments described herein is to affix a length of suture to the mesh at a location corresponding to each distal strut 300A and pass the suture up through each hollow leg 300 into the needle channel 120. In this manner the mesh 200 or other prosthetic device is flexibly coupled to the repair instrument 60 by the suture linear fixation element, but is not directly coupled to the instrument legs 300. Flexible coupling desirably allows the surgeon to maneuver the prosthetic device mesh 200 independently from the instrument 60.

### Overview of Method of Using the Hernia Repair Instrumentation

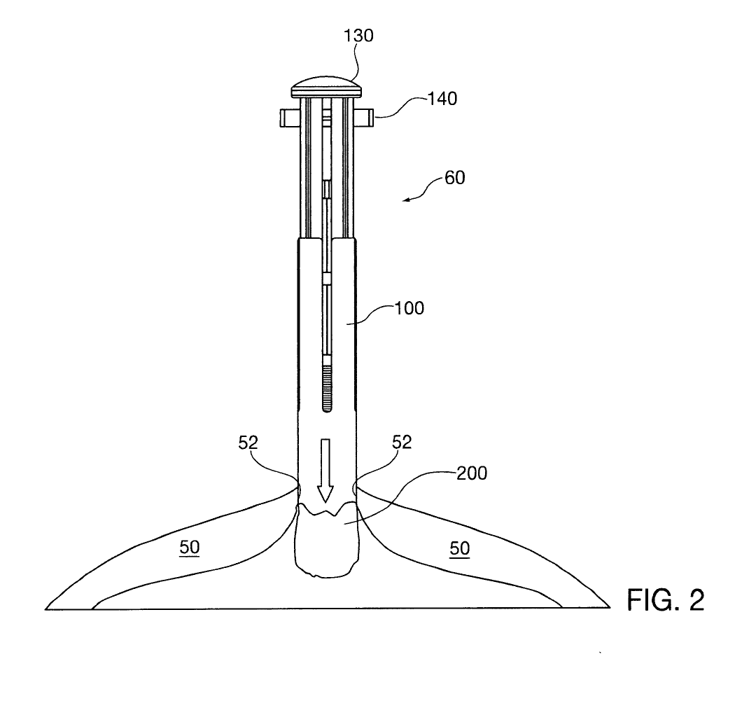

FIG. 2 shows schematically placement of the hernia repair instrument 60 within a patient's abdominopelvic wall 50. For simplicity of the figure various layers of tissue which constitute the abdominal wall are not shown.

Prior to insertion of the repair instrument 60, the operating physician would prepare the hernia site by retracting any tissue, such as bowel or intestine, from the herniation site and restore it to its normal position within the abdominal cavity. Preparation is performed with known laparoscopic or other techniques, including at the discretion of the physician pressure inflation of the patient's abdominal cavity to create volumetric spacing between the patient's viscera and the inner abdominal wall peritoneal layer. After site preparation, the physician incises the patient's abdomen directly within the herniation site, in order to create a dissection in communication with the abdominal cavity. Referring to FIG. 2, ideally the incision 52 is slightly smaller than the outer diameter of the device body tube 100, in order to maintain an airtight seal between the instrument 60 and the patient for inflation of the abdominal cavity. The physician dilates the incision 52 with the rounded end 102 of the device body 100 and inserts the instrument 60 into the patient's abdominal cavity. Insertion instrumentation will be explained in greater detail below. The device body tube 100 is inserted sufficiently into the patient so that the mesh 200 is retained within the abdominal cavity on the inside of the herniation site.

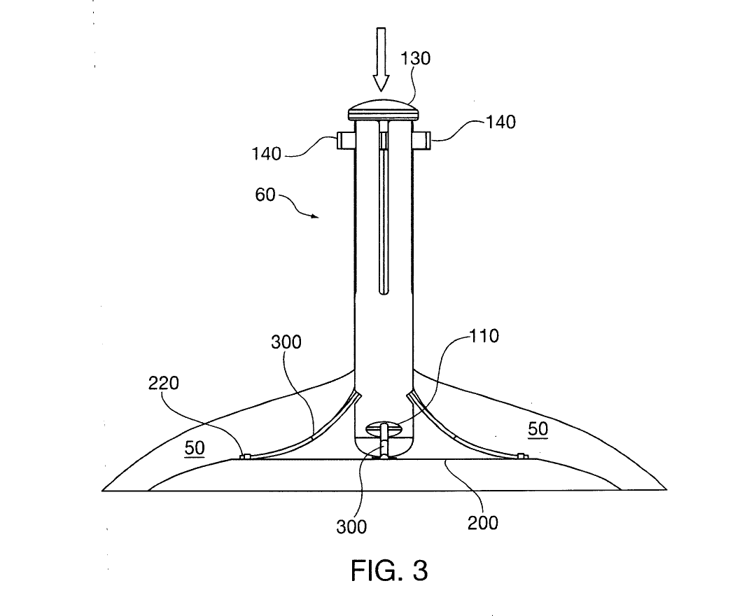

As shown in FIG. 3, the mesh 200 is deployed to its taut position within the patient's abdominal cavity by depression of the plunger 130, thereby extending the device body legs 300 to their fully extended position. Orientation of the device body legs every 90 degrees around the circumference of the mesh 200 biases the mesh to a circumferentially taut configuration for abutment against the patient's abdominal wall 50 to establish a fixation site. In many of the instrument embodiments shown and described herein the prosthetic deployment and fixation site establishment legs 300 or equivalent deploy through a retrograde motion. In their pre-deployment state the legs 300 generally point away from the instrument 60 or equivalent in the direction of device insertion advancement path within the patient. During deployment the legs 300 generally traverse a retrograde course so that their tips are oriented at least at a ninety degree (90°) or greater angle backwards from the insertion direction.

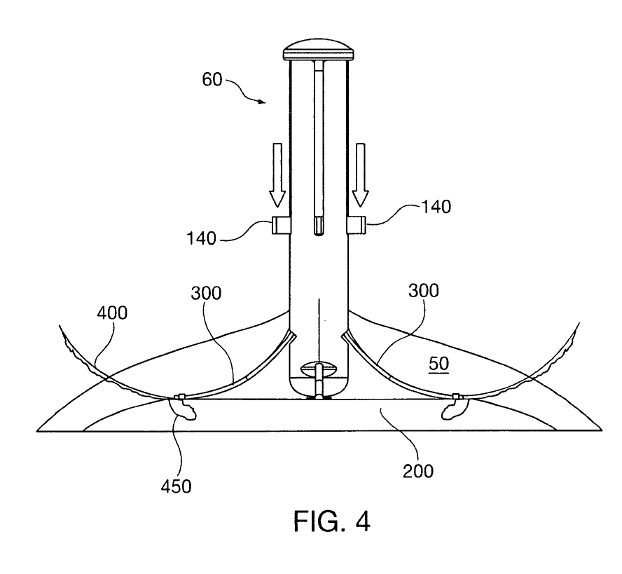

Referring to FIG. 4, the mesh 200 is affixed to the patient's abdominal wall 50 over the herniation site (thereby forming a “patch” over the hernia) by advancing the needle sliders 140 and thereby advancing the needles 400 through the patient's abdomen and outer skin, where they can be retracted by the physician. The needles 400 traverse a retrograde course, generally backwards from the instrument 60 initial insertion direction, through the patient's body tissue 50. Each of the needles 400 is attached to a corresponding suture 450. The end of each suture 450 distal or opposite the needle 400 is attached to the mesh 200. A fastener could be used to maintain suture 450 under tension such that the mesh 200 is retained in taut contact with the patient's abdominal wall over the herniation site. Alternatively, a pair of sutures 450 can be attached to each needle. A knot may be tied between this pair of sutures 450 or a fastener could be used to maintain sutures 450 under tension, so that the mesh 200 is retained in taut contact with the patient's abdominal wall over the herniation site.

While the exemplary repair instrument 60 shown in FIGS. 1A-4 has four device body legs 300 that are oriented circumferentially about the mesh 200 at 90 degree intervals, additional device body legs 300 may be employed if it is desired to orient more than four needles 400 around the mesh 200 at less than 90 degree angle circumferential spacing around the mesh marginal edge. At the physician's discretion additional secondary fixation may be added around the periphery of the mesh through known techniques already practiced in laparoscopic surgery or through additional methods disclosed herein. The physician retracts the needles 400 and device body legs 300 back into the instrument 60 by retracting the respective needle advancers 140 and plunger 130. Thereafter the device body 100 may be withdrawn from the patient's incision 52. The incision 52 is closed using known techniques.

While FIGS. 1-4 have set forth general descriptions of the instruments and their method of use of the present invention for a ventral hernia repair application, the structure of the instruments and exemplary alternative embodiments are described in the remaining text below, including reference to the drawings. Ones skilled in the art can appreciate that the instruments and methods of the present invention can be utilized to implant other types of prosthetic devices in patient tissue.

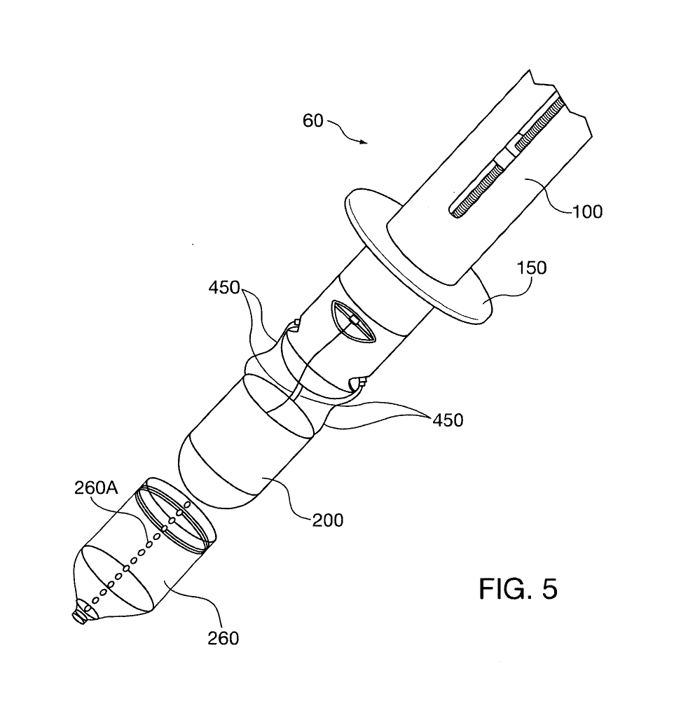

In FIG. 5 the instrument 60 includes an insertion depth stop 150 that may be employed by the physician to limit selectively axial insertion into the patient's hernia incision. When the device body legs 300 are extended, an insertion depth stop 150 is used to compress abdominal wall 50 against said legs to provide additional device stability during mesh fixation. In this embodiment, the repair mesh 200 is folded to fit around the circumference of the instrument device body 100, and may be covered by a mesh covering sheath 260. The sheath 260 may be fabricated from plastic material and may include formed perforations. Advantageously the sheath 260 may be split open along the perforation line 260A to enable easier removal from the hernia incision after device body 100 insertion. Thereafter the mesh 200 can be deployed within the patient's abdominal cavity. Sutures 450 are attached to the mesh 200 circumference at 90 degree intervals corresponding to each of the device body tubes and needles (not labeled in this figure).

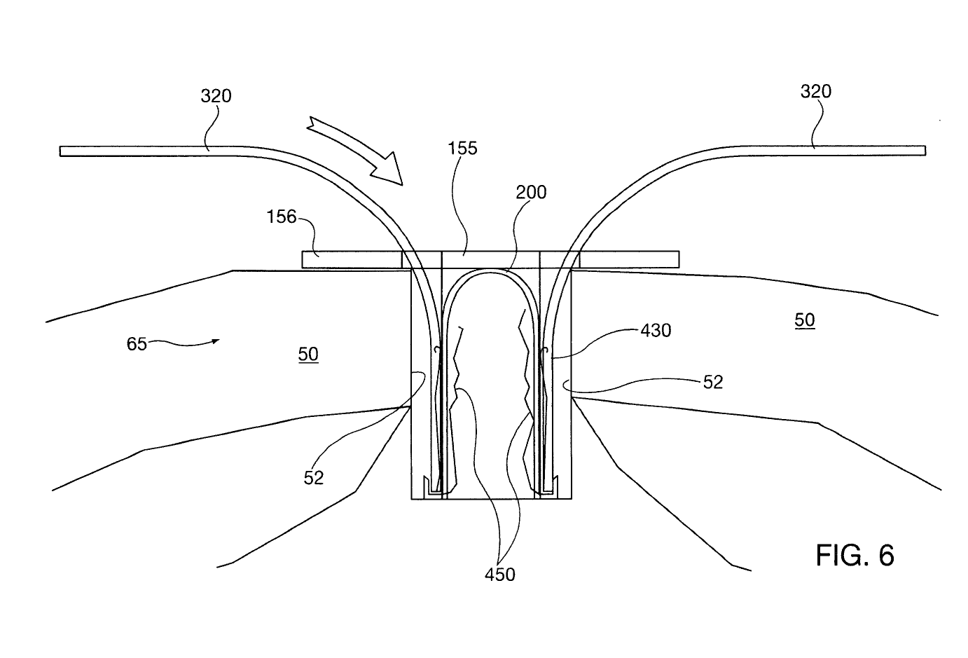

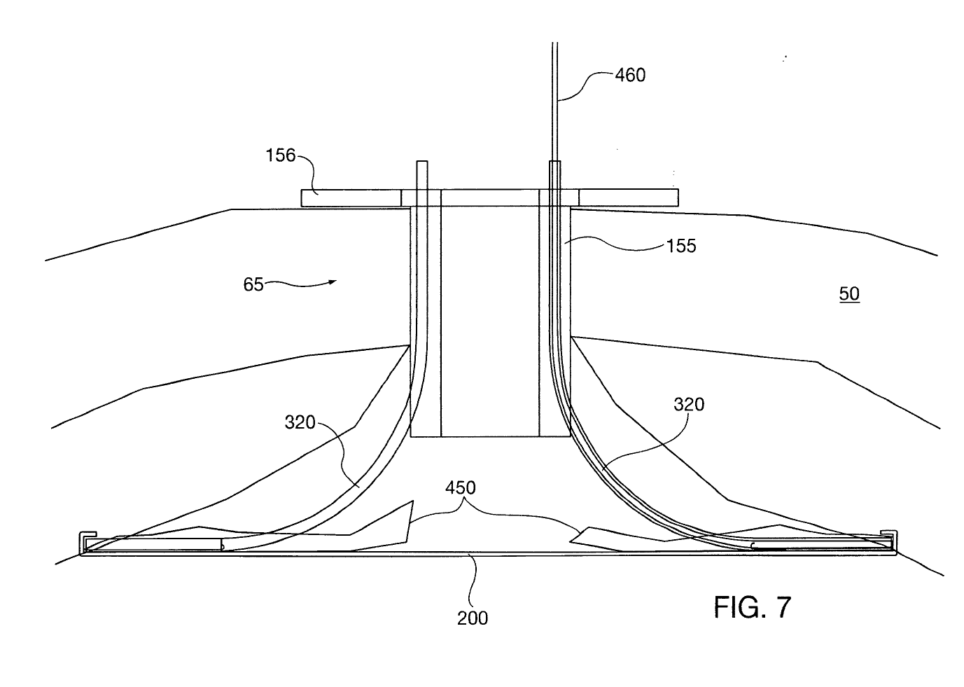

An alternate embodiment of the instrumentation of the present invention is the L-shaped device body 65 shown in FIGS. 6-8. Port device body 155 does not have a plunger, and is shown having an insertion depth stop 156. In this embodiment the mesh 200 is retained within the bore of the device body 155 and dropped into the abdominal cavity after insertion of the device body port into the patient. Four L-shaped cannulae 320 are serially inserted into the device body port 155. When an individual L-shaped cannula is advanced into the port 155, the distal end is oriented generally normal to the port 155 bore axis at a tangential angle relative to the patient's abdominal wall 50 of 90 degrees or greater retrograde motion. This deploys and tauts the mesh 200, as is shown in FIG. 7. A needle driver 460 is inserted in each of the L-shaped cannulae 320, to advance the needles 400 through the full thickness of the patient's tissue. The physician retracts the needles 400 and tensions the sutures 450.

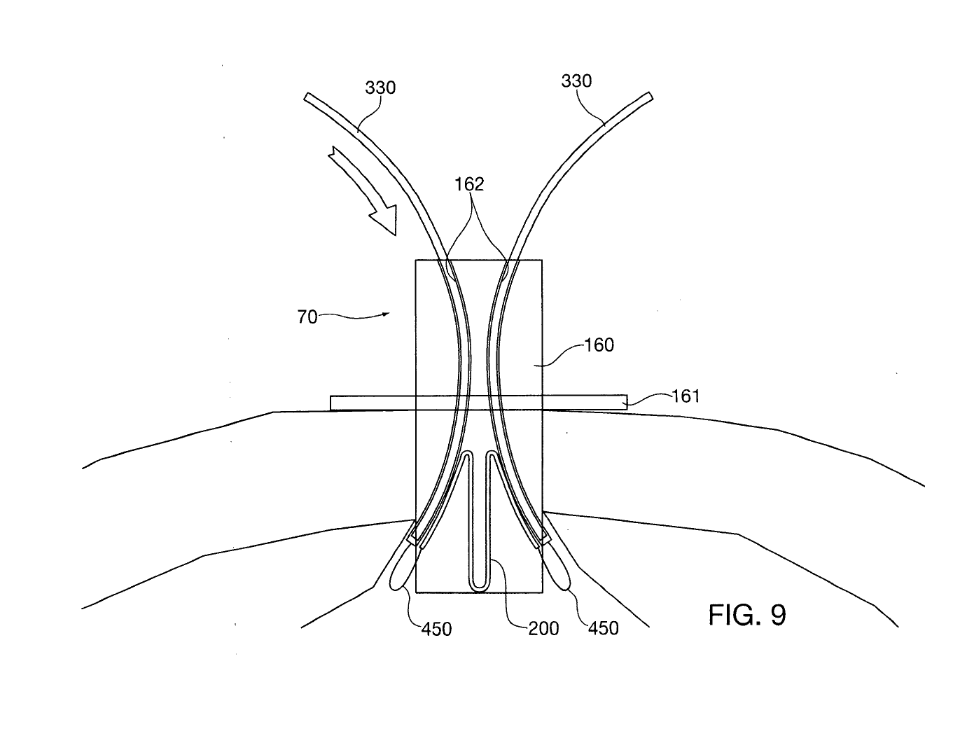

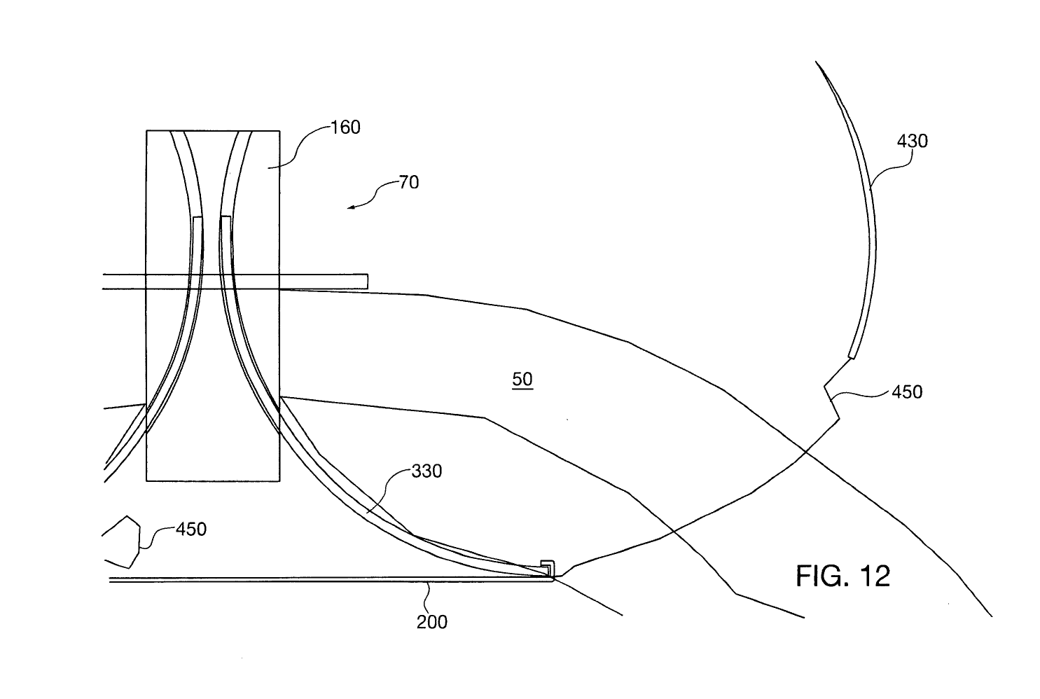

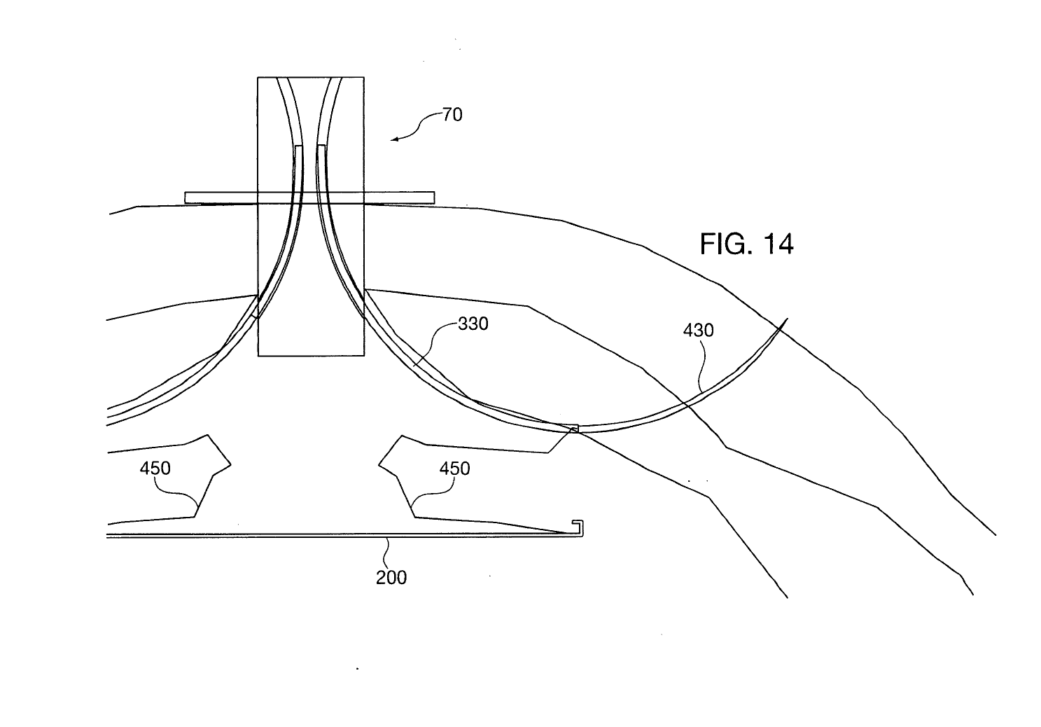

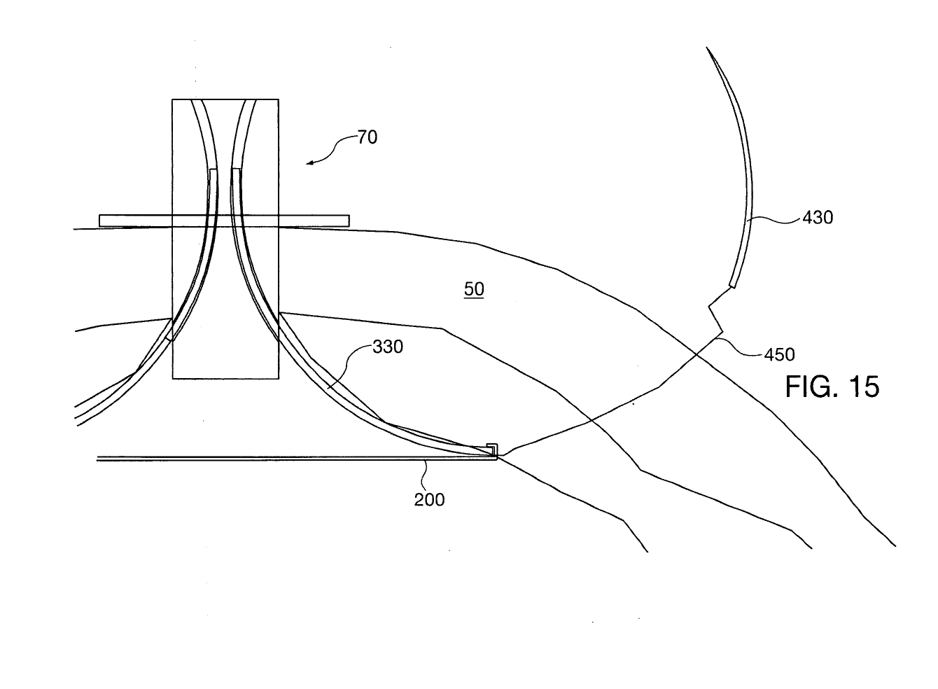

FIGS. 9-15 show a constant radius needle is instrumentation embodiment 70. This embodiment utilizes a cannula port 160 with insertion depth stop 161. The cannula port 160 has an inner bore that defines needle tracks 162 having a curvature radius and diameter adapted for receipt and guidance of constant radius cannulae 330, oriented at 90 degree circumferential positions. The cannula port 160 retains pre-deployment configuration repair mesh 200, shown schematically in FIG. 9. The mesh sutures 450 respectively are threaded into one of the corresponding constant radius cannulae 330. As was described with respect to prior embodiments, the distal end of each suture 450 is affixed to the mesh 200 circumference and the proximal end is coupled to a needle. In this embodiment constant radius needles 430 are utilized, having a profile conforming to the constant radius cannulae 330.

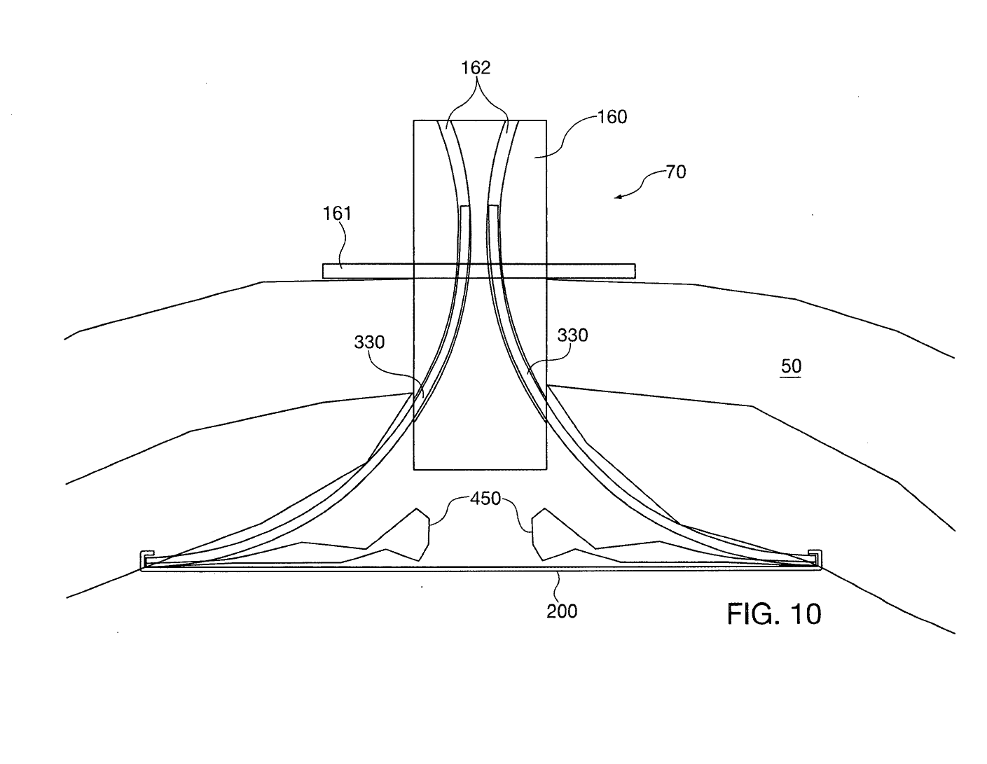

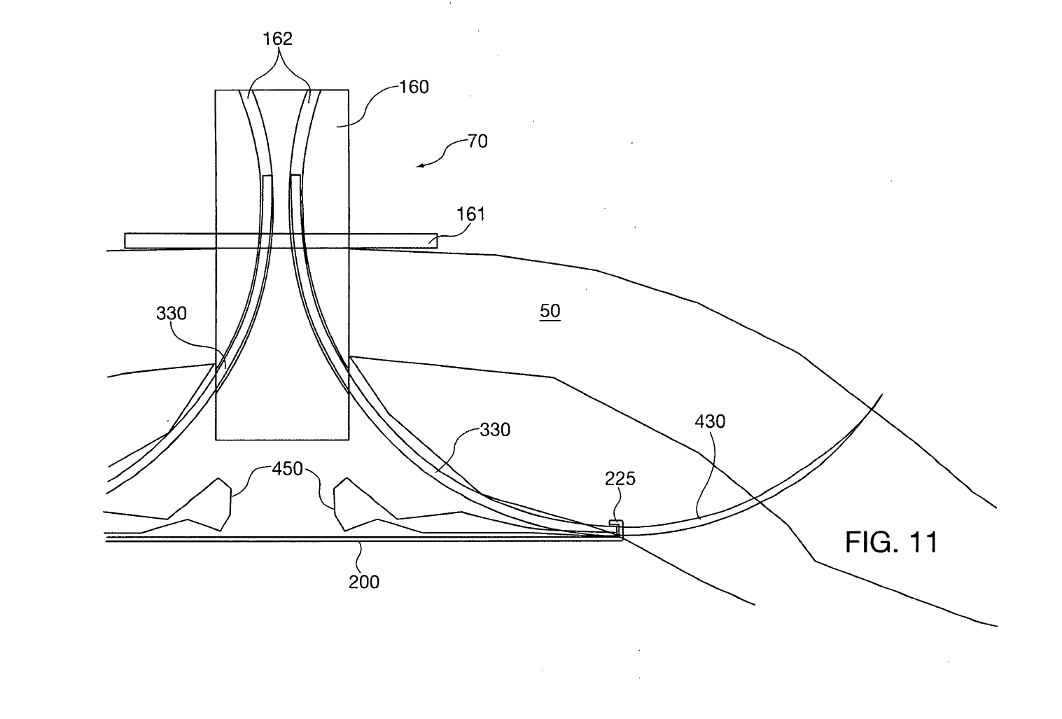

For illustrative purposes, two mesh deployment and primary fixation site alignment apparatus and methods are shown for the constant radius needle instrumentation embodiment 70, but these methods are applicable for many of the other disclosed embodiments. In FIGS. 10-12, the mesh 200 circumferential rim 225 has a C-shaped cross section that engages distal ends of the constant radius cannulae 330 (see detail in FIG. 11) in the pre-deployment position. Desirably the circumferential rim may also incorporate a resilient band or hoop (not shown) that matches the circumferential profile of the mesh. Such a resilient hoop aids desired circumferential expansion tensioning of the mesh 200. When the cannulae 330 are advanced in the tracks 162 their constant radius causes their distal tips and the engaged mesh 220 to transverse tangential and in retrograde (generally backwards) fashion relative to the cannula port 160 initial insertion/advancement orientation toward the patient's abdominal wall 50 and abut against it at the maximum advancement position to establish a prosthetic device fixation site. Thereafter, the constant radius needles 430 are advanced through the cannulae 330 and passed through the patient's abdomen in a retrograde motion path. The surgeon then pulls the needles 430 out of the patient and tensions the sutures 450 so that the mesh 200 abuts tautly against the patient's inner abdominal wall peritoneal layer.

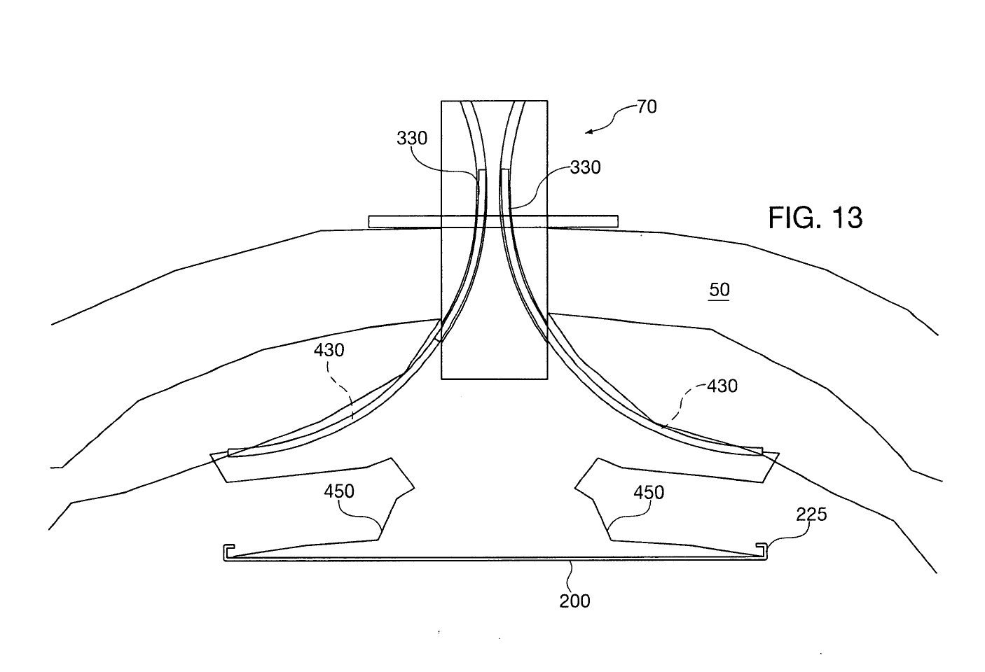

An alternative mesh deployment and implantation site alignment/fixation method is shown in FIGS. 13-15, again for illustrative purposes disclosed with respect to the constant radius needle instrumentation 70 but applicable to other embodiments. In this embodiment the mesh 200 is not directly engaged circumferentially by and supported by the prosthetic implantation site alignment cannulae 330 within the mesh rim 225 or any other direct supportive coupling during deployment, but rather is deployed loosely over the patient's viscera (e.g., bowel) by dropping it in place.

By analogy, the deployed mesh 200 hangs as an inverted parachute, remaining coupled to the instrument 70 by the sutures 450 passing through respective cannulae 330 bores. The use of linear fixation elements such as sutures 450 for flexible rather than direct coupling of the prosthetic device repair mesh 200 to the implantation site alignment cannulae 330 affords the surgeon greater flexibility to maneuver the instruments and mesh by, for example, increasing or decreasing slack in the suture or with other endoscopic instruments. In this embodiment, the constant radius cannulae 330 are advanced tangentially in retrograde fashion relative to the instrument initial advancement path into the patient's tissue, in order to make contact with the patient's abdominal wall tissue 50. The contact point between an individual cannula 330 tip and the patient tissue 50 establishes an implantation fixation site for a primary fixation device. Thereafter, the needles 430 are advanced through the patient's abdominal wall tissue 50 in retrograde fashion, as was done with the prior embodiment of the instrument 70 discussed above. As the surgeon tensions the sutures 450, the circumferential edges of the mesh rim 225 (or for that matter any other supported or unsupported, flaccid, rimless mesh or other prosthetic device) are drawn against the patient's abdominal wall peritoneal layer at each fixation site previously established by the respective cannula/strut/leg 330 tip.

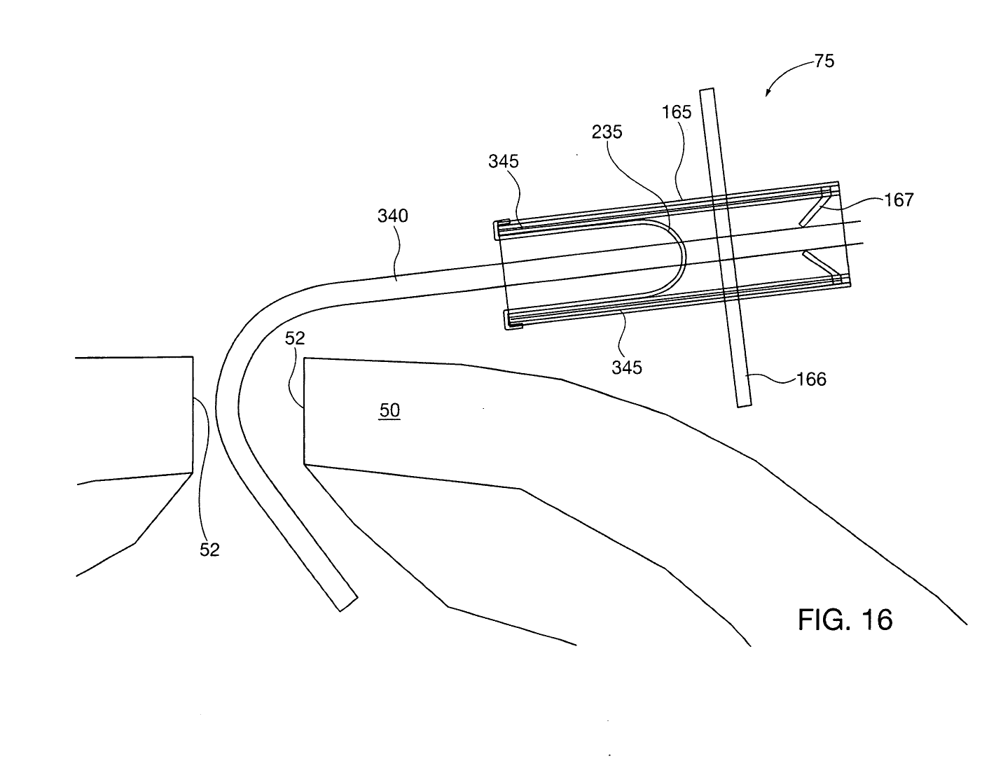

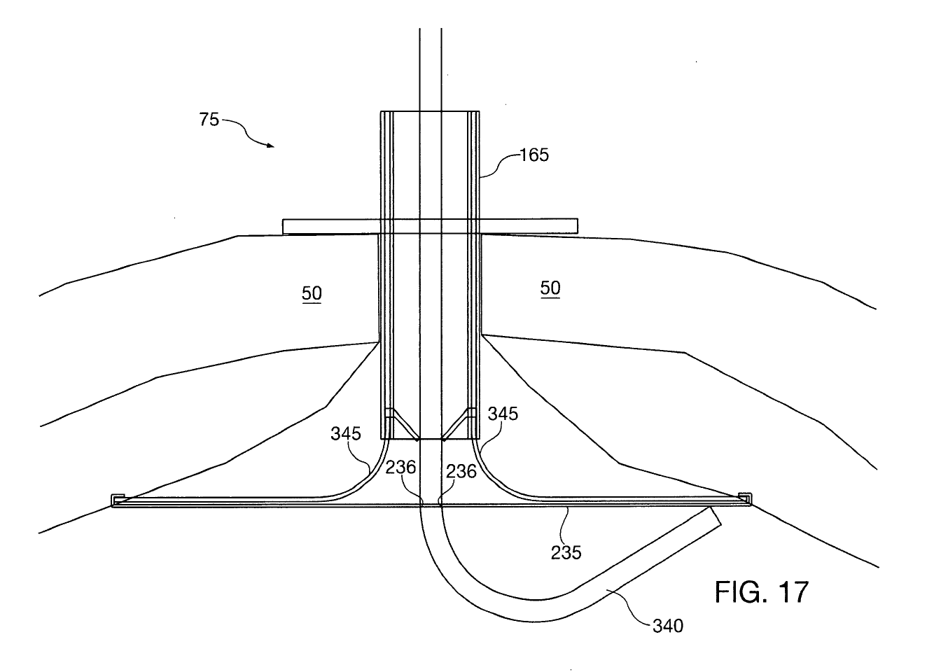

FIGS. 16 and 17 show an indexing cannula instrument 75 embodiment that allows selective radial orientation about the circumference of the hernia incision 52. This instrument embodiment reduces the need to insert additional lateral cannulae into the patient's abdominal cavity for repair mesh fixation instruments. The indexing cannula instrument 75 has a generally V-shaped indexing cannula 340 capable of 360 degree circumferential rotation when advanced into the patient incision 52. Thereafter, indexing cannula port 165 is inserted into dilated incision 52 forming a tight seal therein. Adjustable stop 166 allows the physician to limit insertion depth of the cannula port 165. Seal 167 preserves airtight seal in the bore void between the indexing cannula 340 and the port cannula 165. Repair mesh 235 is retained within the port cannula 165 and has a central aperture 236 (e.g., of round or slit configuration) that allows through passage of the indexing cannula 340. The mesh aperture 236 is of a sufficiently small diameter to prevent ex-filtration of the patient's visceral tissue. If desired, the repair mesh aperture 236 may be sealed after retraction of the indexing cannula 340, such as by a suture or a covering flap (not shown). Repair mesh is circumferentially coupled to flexible shape memory metal support rods 345 that are slidably coupled to the interior of the port cannula 165. A suitable memory metal is so-called nickel-titanium NITINOL®. As the memory metal support rods 345 are slidably advanced into the patient they assume their generally V-shaped relaxed state, oriented tangential to the port cannula 165 bore axis and thereby radially expand and circumferentially tension the support mesh to a taut configuration in abutment with the patient's peritoneal abdominal wall layer. Thereafter, mesh fixation instrumentation and anchors (for example sutures, needles and needle passers) may be passed through the indexing cannula 340 bore. The indexing cannula 165 is oriented radially in any desired position where the surgeon wishes to anchor the mesh 235 circumferential fixation anchors.

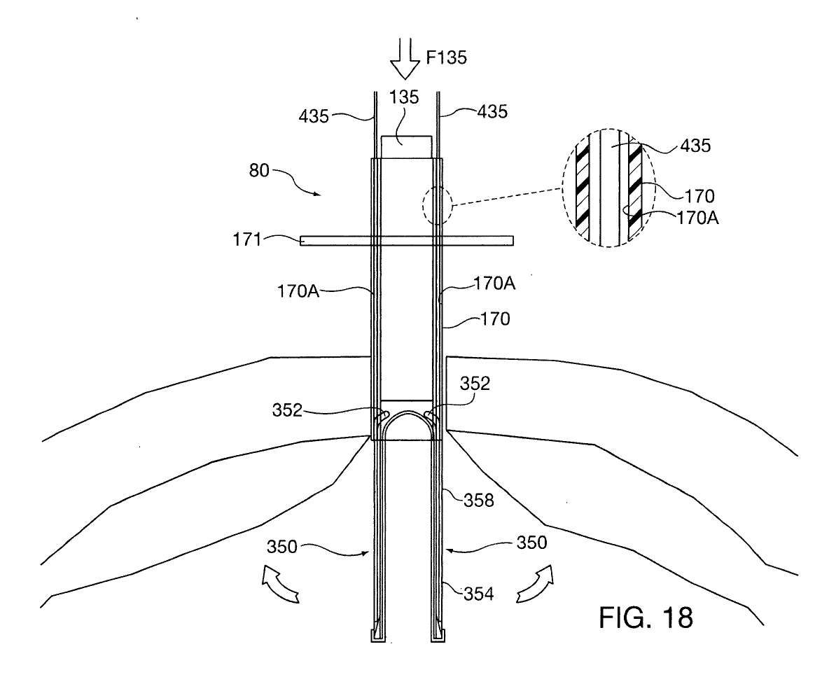

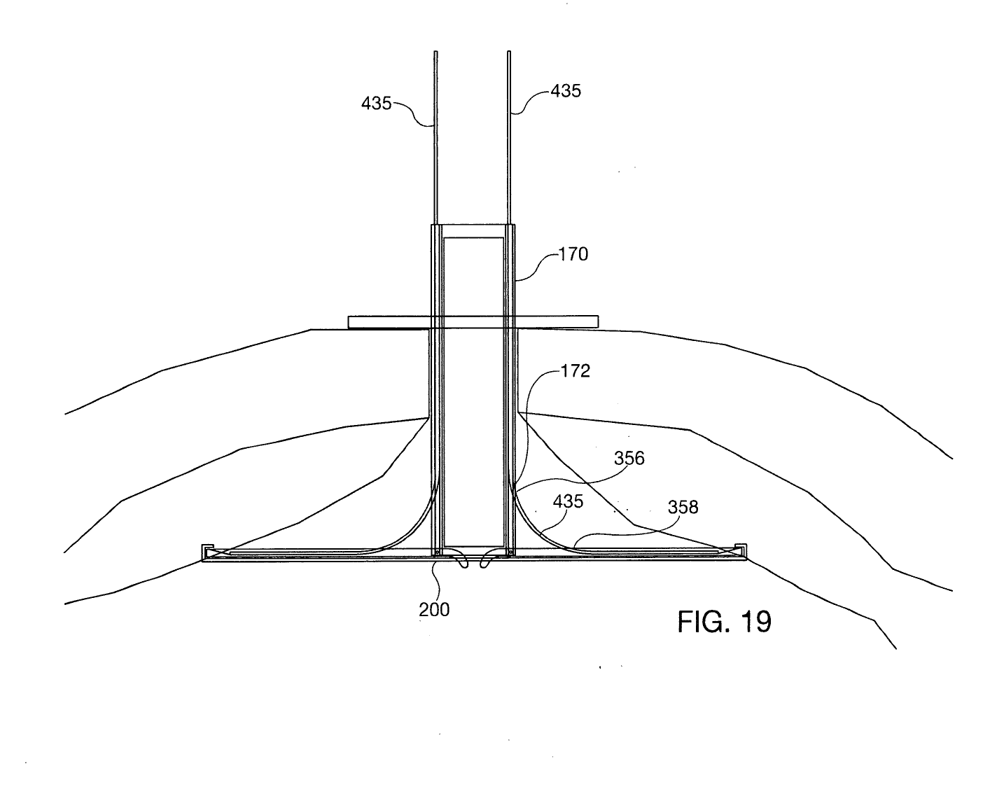

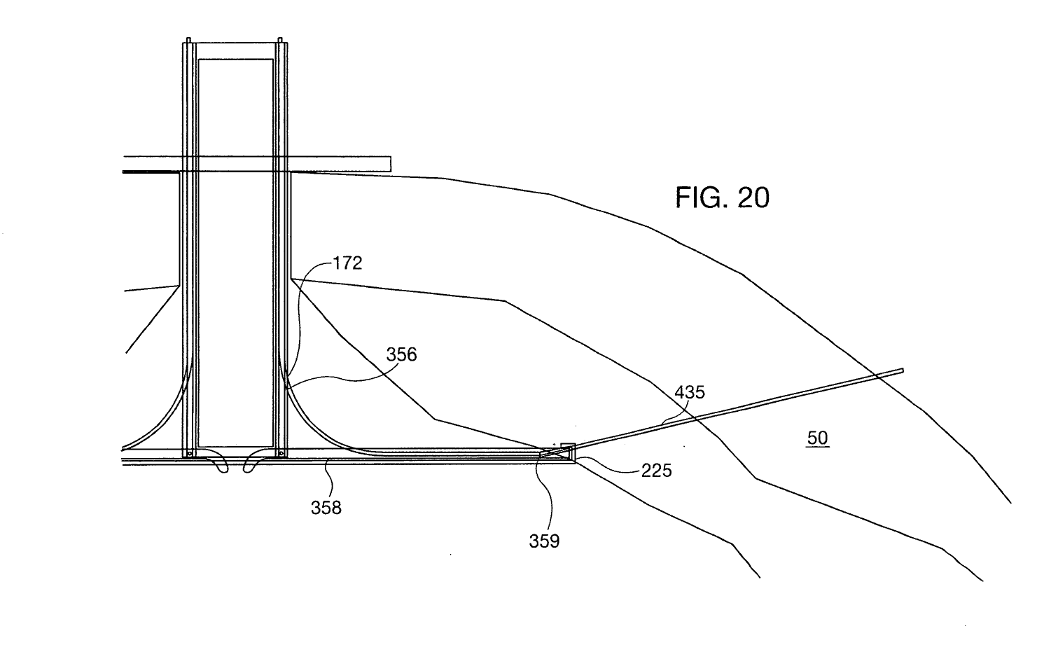

A lever arm cannula instrument 80 embodiment is shown in FIGS. 18-20, that provides desired taut circumferential deployment of repair mesh 200 in a relatively small volume storage package. The instrument 80 has a cannula port 170, adjustable insertion stop 171 and circumferential axially oriented relief cuts 172. The cannula port 170 has four central bores 170A formed in the port 170, in which it retains four cannula lever arms 350 oriented circumferentially at 90 degree angles, as was described with respect to other embodiments. The lever arms 350 may be reciprocable within each bore 170A from a collapsed position captured within the port 170 to an extended position projecting from the port. Alternatively, the lever arms 350 may be affixed at the distal end of the port 170 so that they are always extending therefrom.

Each lever arm 350 has a central bore, an axial relief cut 356, a lever cam 352 and lever 354 pivotally affixed thereto. The distal end of each lever 354 engages the deployable repair mesh 200, such as by the C-shaped circumferential rim 225 previously described. Each lever 354 is oriented coaxial with the port relief cuts 172, and defines an axial lever relief cut 358 in communication with the port relief cut. Depression of push rod 135 within the cannula port 170 central bore cams open the levers 354 tangentially to the bore and thereby circumferentially expanding and tensioning the repair mesh 200. All three relief cuts 172, 356 and 358 are axially aligned so that bendable full-length needles 435 may be advanced through respective corresponding central bores 170A formed in the port 170 and in the lever arms 350. Ramped surface 359 defined by the distal tip of the lever 354 deflects the needle 435 generally tangentially into the patient's abdominal wall 50 so that tensioning of a suture affixed to both the needle and the mesh 200 enables taut tensioned abutment of the mesh to the patient's peritoneal layer.

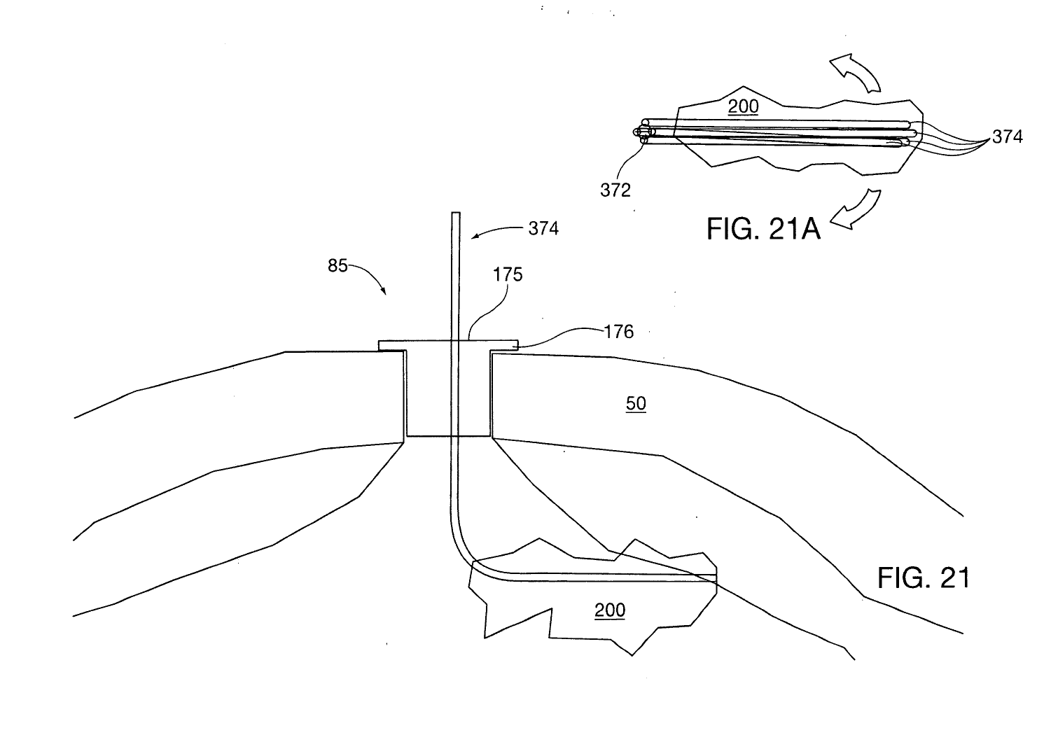

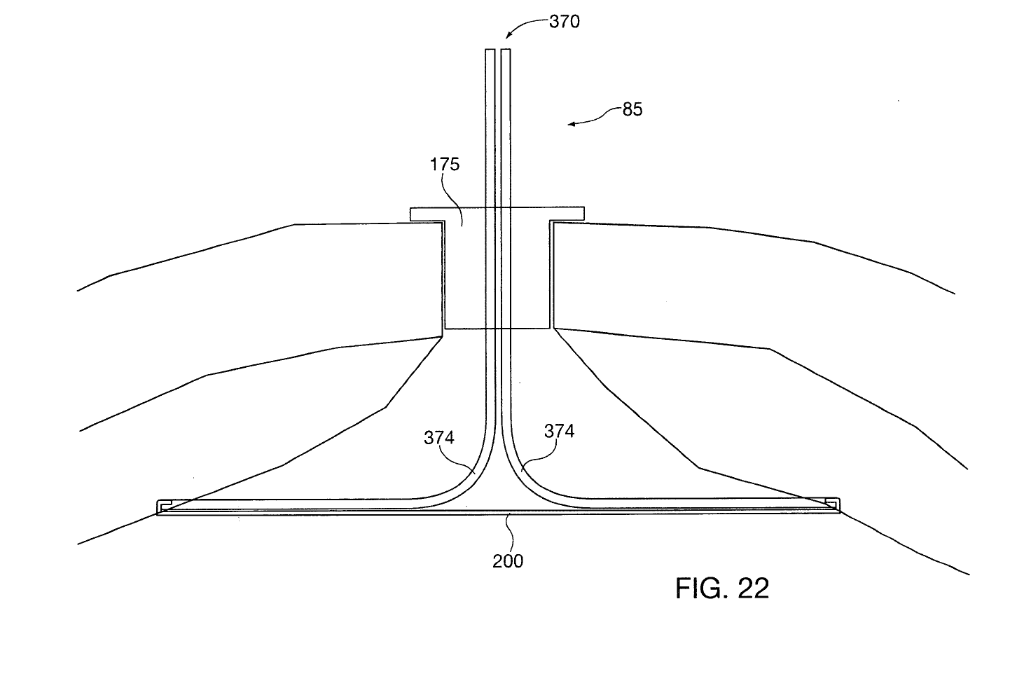

A foldable L-shaped arm instrument 85 is shown in FIGS. 21-22. The instrument 85 has a cannula port 175 and insertion stop 176 with a central bore. Foldable L-shaped arm assembly 170 has a common pivot 372 for L-shaped arms 374. As shown, there are four arms 374 that are affixed to the repair mesh 200 so that when the arms 374 are fan-pivoted from the collapsed position shown in FIG. 21A to the fully extended, mesh deployed position shown in FIG. 22, the deployed arms are oriented at 90 degree positions about the circumference of the mesh. Each of the L-shaped arms 374 is a cannula with an internal bore for passage of mesh fixation instruments.

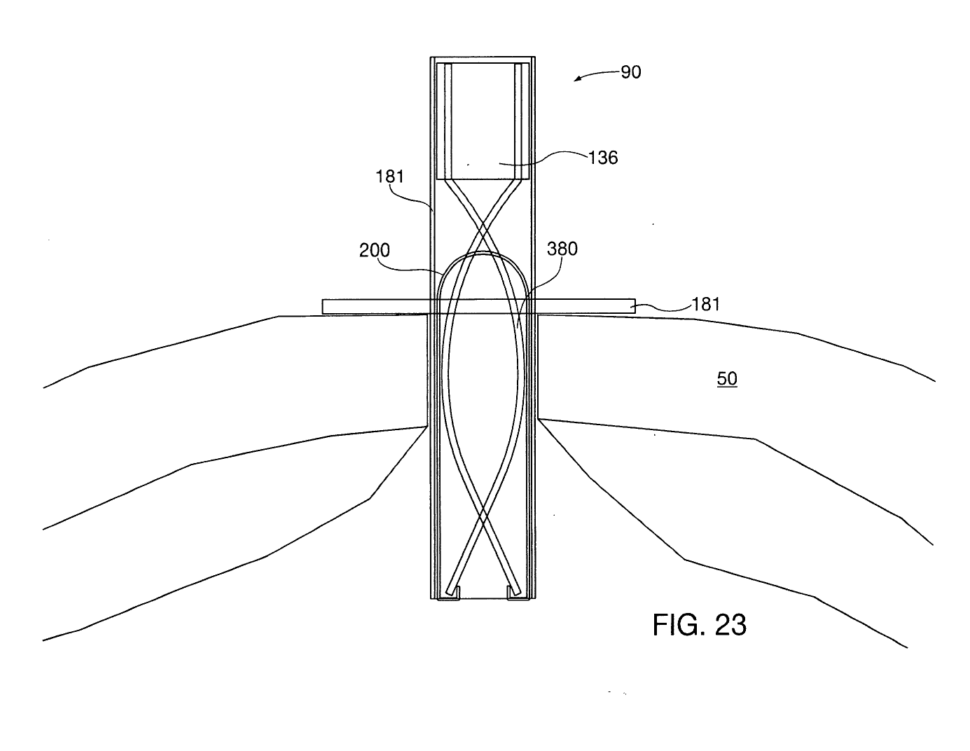

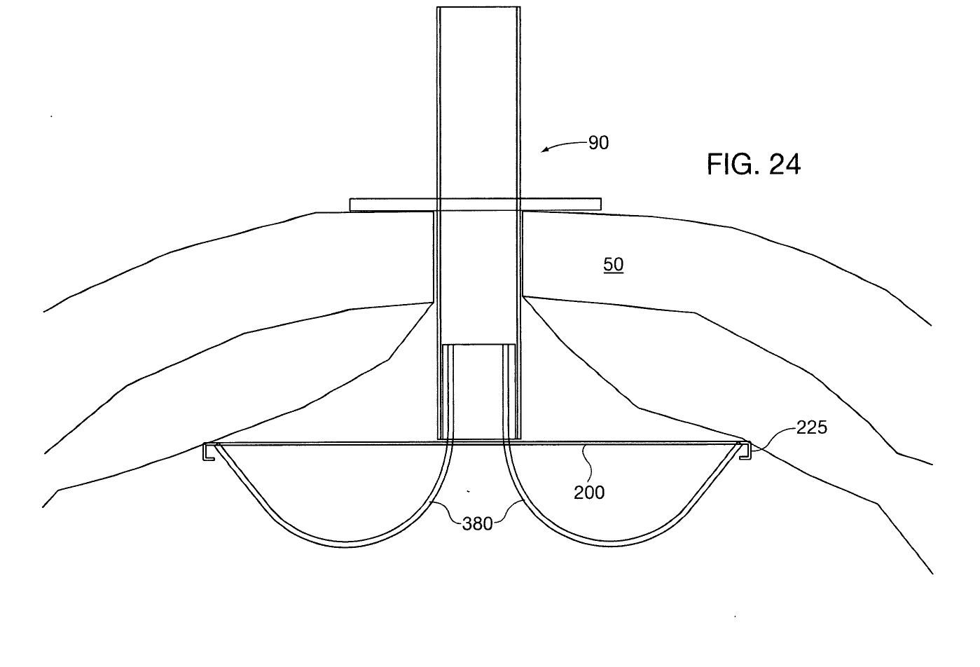

FIGS. 23-24 show a precurved memory metal mesh deployment instrument 90, having a cannula port 180 and adjustable stop 181. The central bore of the port 180 contains pre-deployed repair mesh 200 that is attached to a plurality of shape memory metal alloy cannula tubes 380. A suitable memory metal is so-called nickel-titanium NITINOL®. The memory metal cannula tubes 380 are coupled to a tube carrier 136 also contained within the cannula port 180 that may be depressed to deploy the memory metal tubes and the mesh 200 into the patient's abdominal cavity. When the memory metal tubes 380 are deployed and no longer constrained within the cannula port 180 they assume the shape shown in FIG. 24, thereby deploying the repair mesh to its desired, circumferentially taut configuration abutting the patient's abdominal wall 50. The inner bores of the memory metal cannula tubes 380 provide a passage for mesh 200 fixation instruments, such as needles and sutures previously described.

Hernia repair mesh is commercially available to the medical community in circular or oval configurations of various sizes. Using the prosthetic device deployment, implantation site fixation and primary fixation instruments and methods of the present invention enable implantation of flaccid, unsupported prosthetic devices, such as mesh, as well as devices with self-supporting structure. However, as has been previously discussed, the mesh or other prosthetic device may be configured to include deployment attachment pockets, structural reinforcement circumferential rims or circumferential bands/hoops in order to affix it to exemplary deployment instrumentation embodiments shown herein. It is also possible to construct an inflatable circumferential pocket around the periphery of the mesh that will deploy the mesh in the desired circumferentially taut configuration for affixation to the patient's abdominal wall peritoneal layer. Thereafter the pocket is deflated. Alternatively an auxiliary inflation device can be interposed between the mesh and the patient's viscera within the abdominal cavity and thereafter removed after mesh affixation to the peritoneal layer/abdominal wall.

### Primary Prosthetic Device Fixation

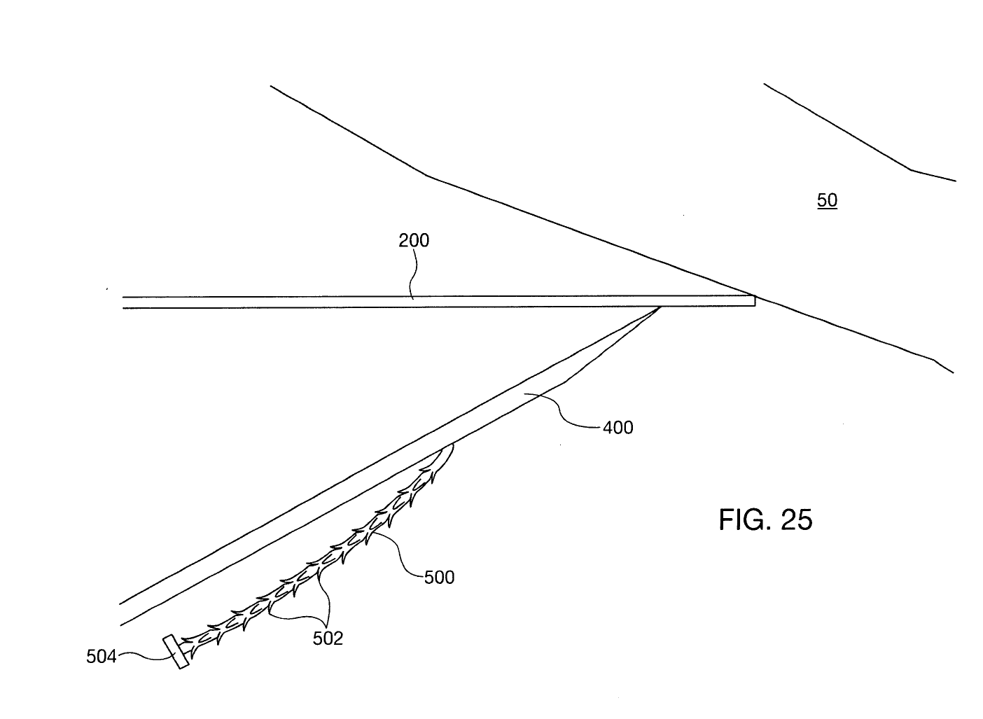

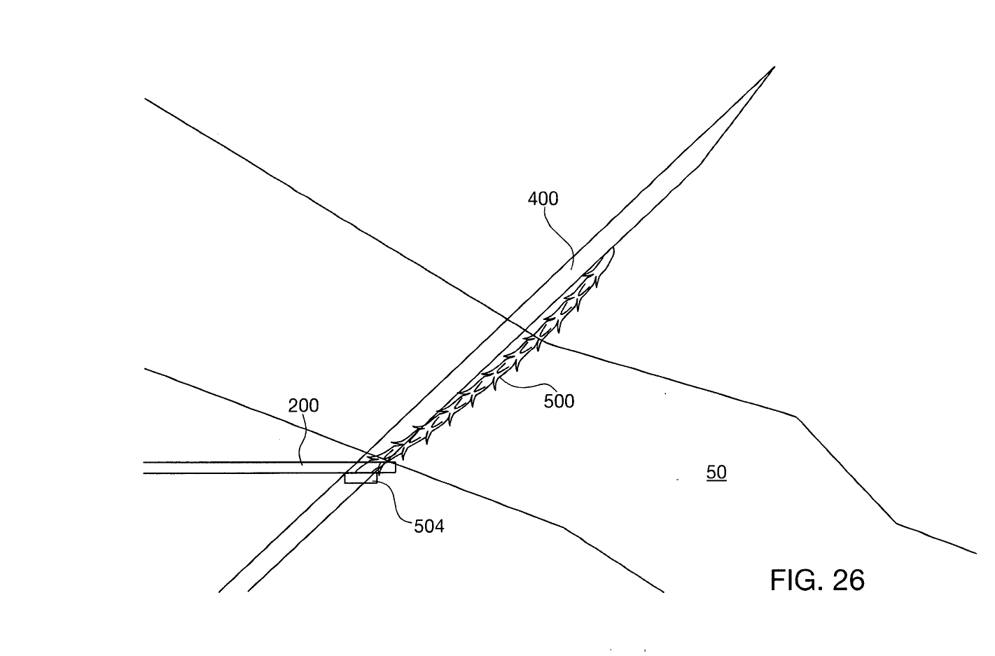

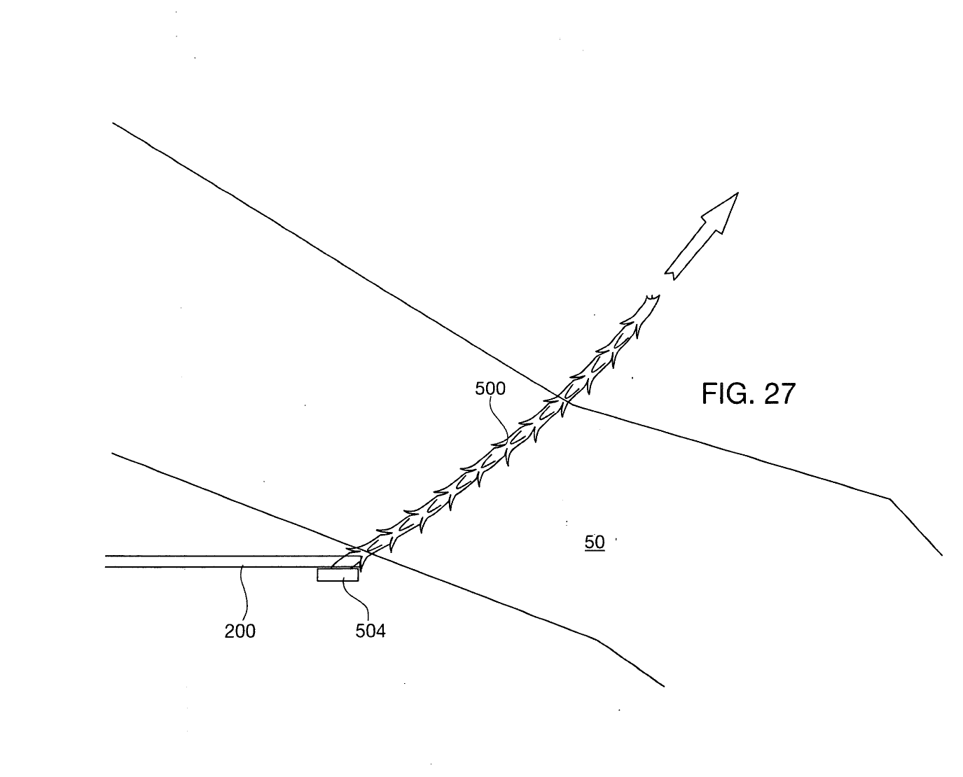

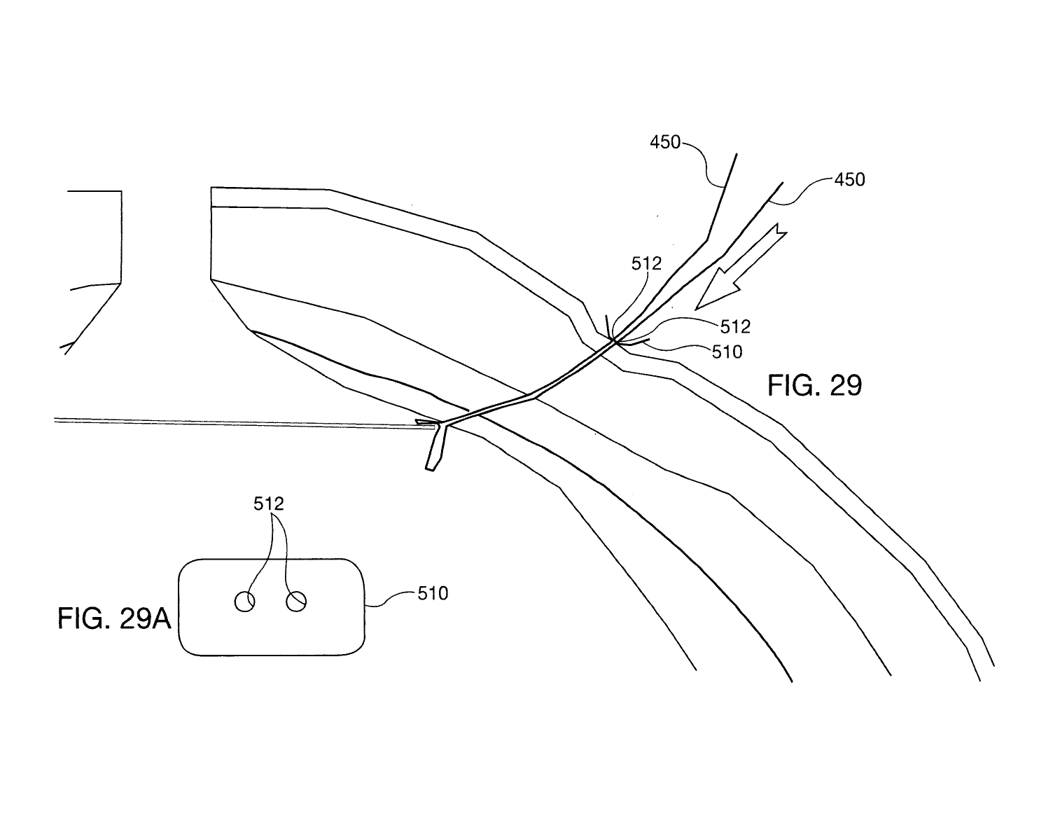

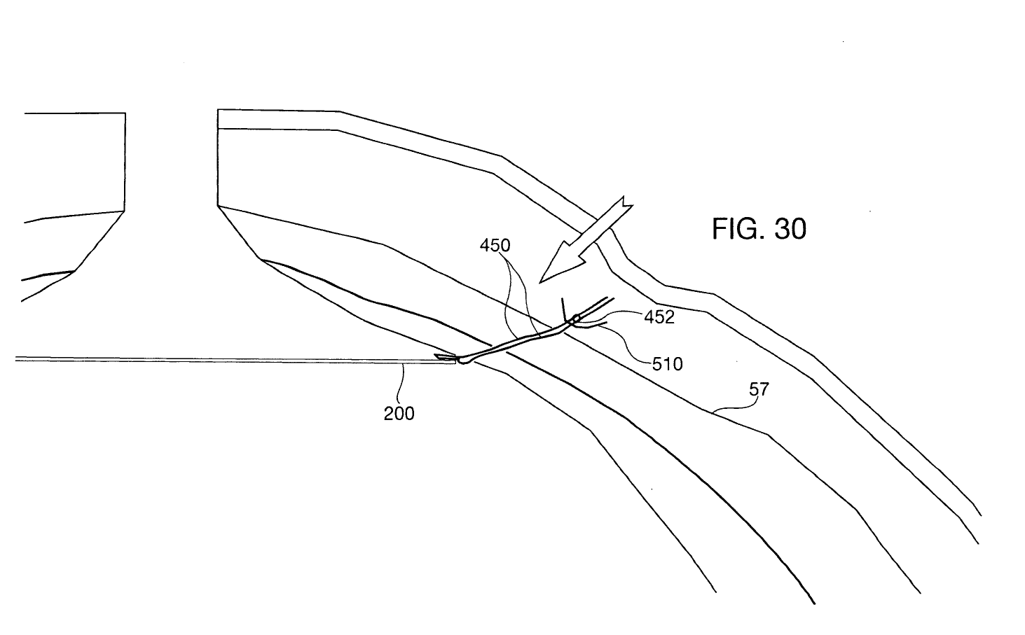

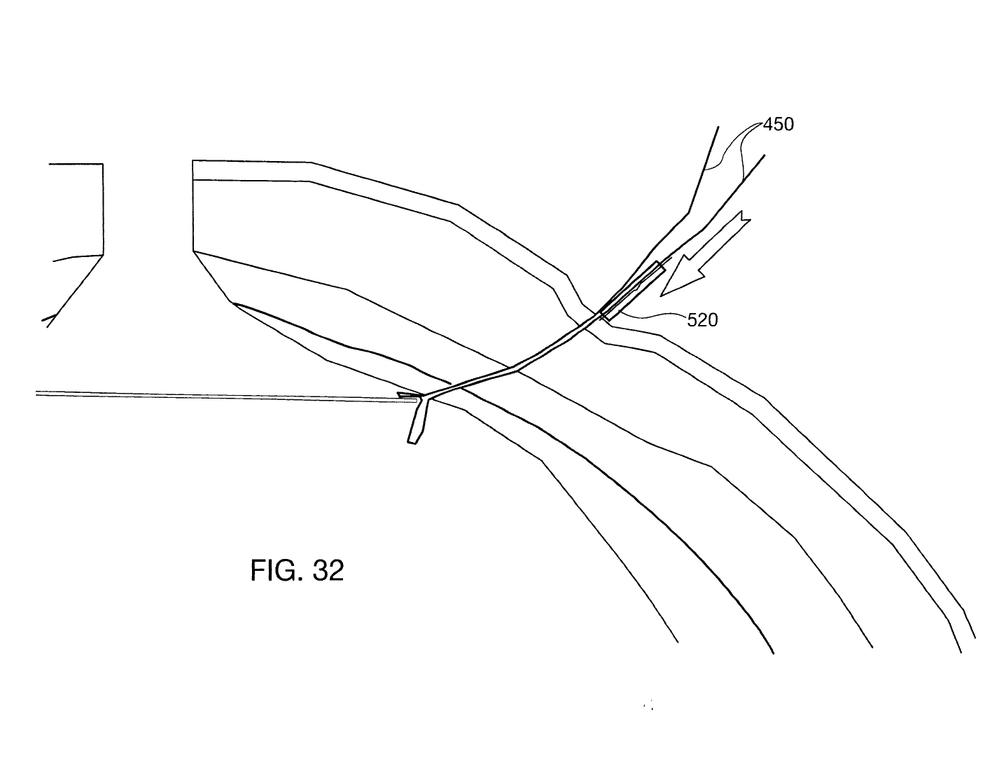

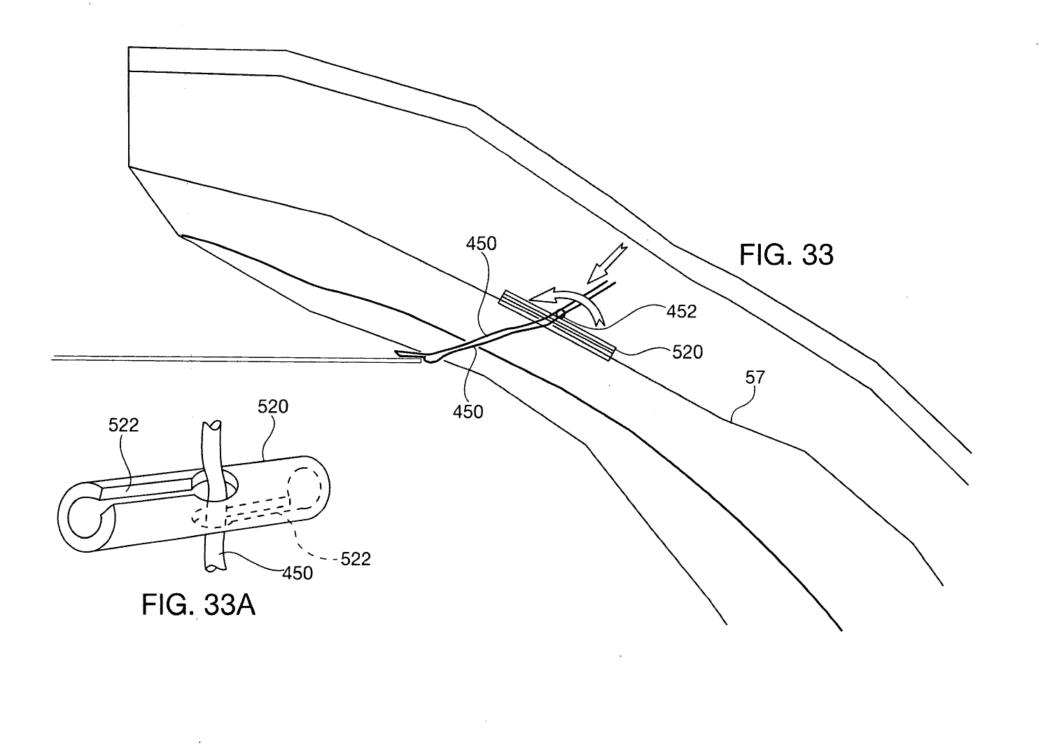

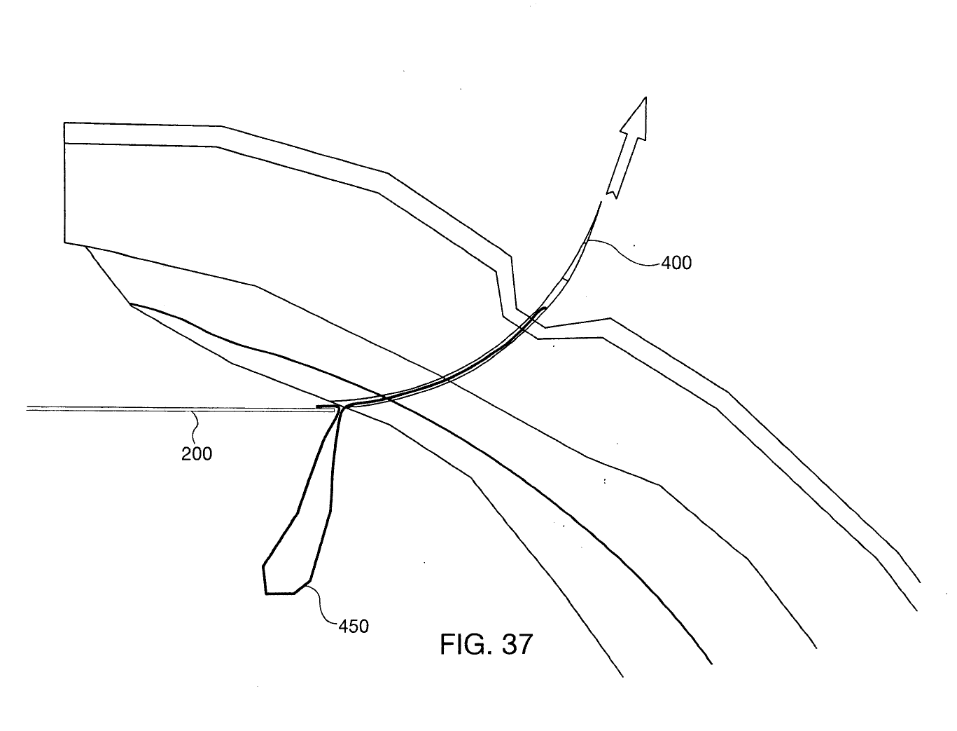

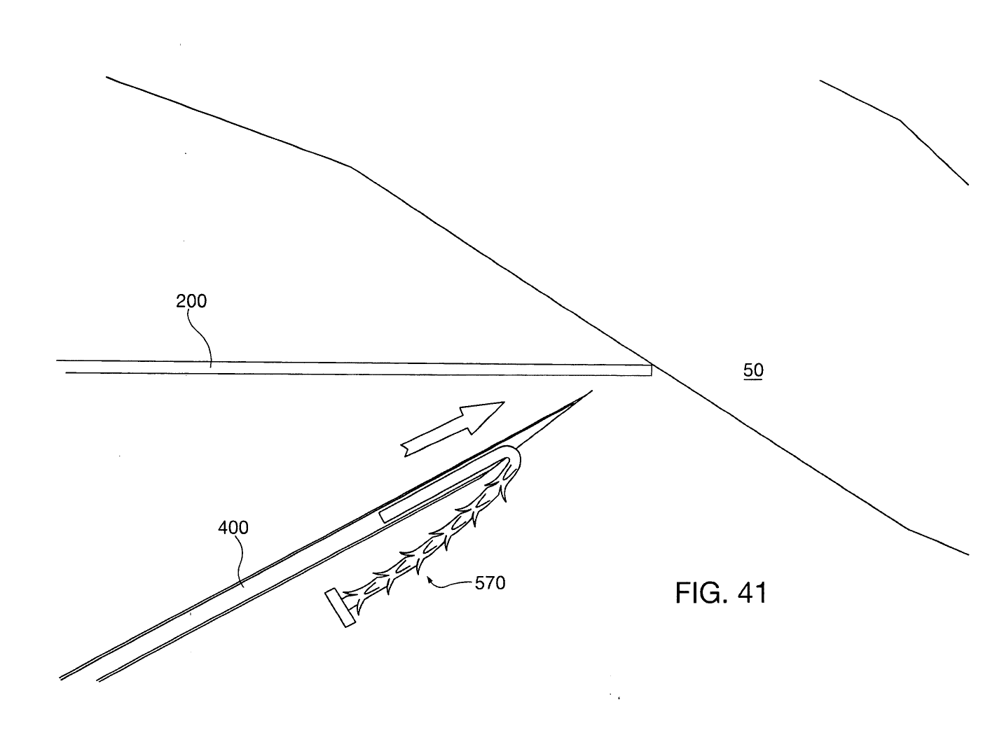

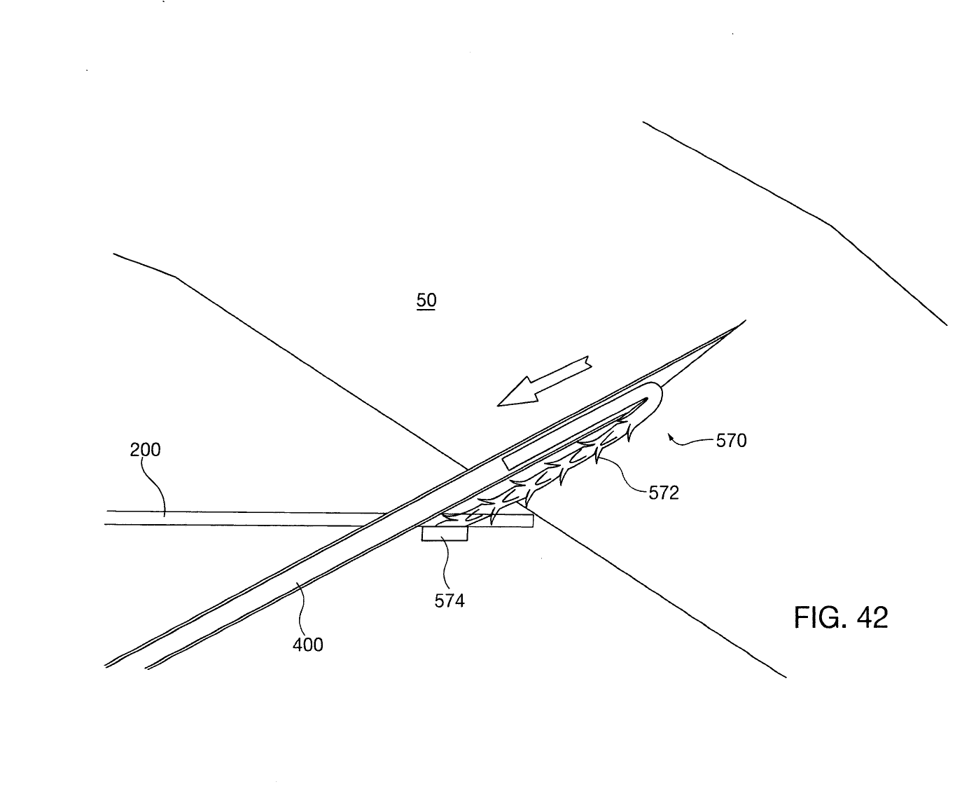

FIGS. 25-45 show methods and apparatus to affix hernia repair mesh 200 to a patient's abdominal wall 50. While many of the instrument embodiments previously described have oriented deployment and fixation cannulae at four ninety degree radial positions about the repair mesh circumference, more or less fixation points can be chosen by the physician, so long as desired mesh or other prosthetic device circumferential tension and abutting integrity with the patient's tissue (e.g., abdominal wall) are achieved. It may be desirable to achieve primary fixation of the mesh with at least four fixation points oriented at ninety degree radially offset positions and to utilize secondary fixation points between the primary fixation points.

In FIGS. 25-27 the circumferential peripheral edge of the mesh 200 is shown in desired circumferentially taut, abutting relationship with the patient's abdominal wall, so as to reduce likelihood of future visceral ex-filtration around the edges of the repair mesh or centrally through the existing, repaired herniation site. Affixation needle 400 of known construction retains barbed suture 500 and passes the suture through the mesh into the patient's abdominal wall. The barbed suture 500 has integrally formed barbs or quills 502 that engage surrounding tissue and inhibit axial withdrawal of the suture. During suture 500 advancement, the barbs 502 are urged generally flat along the suture body circumference, but the barbs spring out and resist suture retraction by engagement of their pointed, distal ends with surrounding tissue. The suture 500 has a suture stop 504 that may be integrally formed therein or may be a separate component, such as an annular ring, that is threaded over the suture and captured by knot, crimp, bonding, weld or other affixation technique method. The barbed suture may be passed through the patient's abdomen transdermally and tensioned. Thereafter the suture 500 may be cut to desired length by the surgeon.

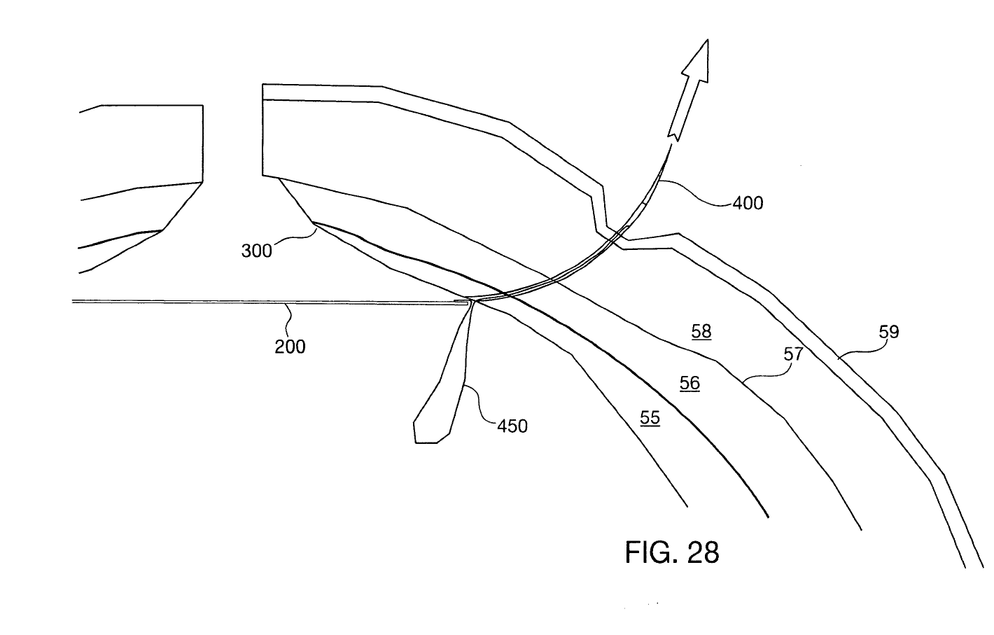

FIGS. 28-39F show additional primary and/or secondary mesh affixation apparatus and techniques that employ a radially projecting fastener 510, 520, 530, 540 that abuts the patient's outer fascia 57 and reduces the likelihood that the mesh 200 will separate from the desired taut abutting orientation with the patient's peritoneal layer 55. In each of these fastener embodiments to be further described herein an axially tensioned suture body is captured at one end by firm affixation to the repair mesh and at the other end by the fastener. In FIGS. 28-30 the fixation fastener is a pledget 510, a flexible sheet anchor constructed of polymer or fabric, having openings 512 for receipt of suture legs. In this embodiment, a suture 450 is advanced from the patient's abdominal cavity through all intervening layers of tissue and delivered outside the patient's skin. The pledget 510 is inserted transdermally into the patient by capturing the suture 450 within the pledget openings 512. The surgeon forms and advances a suture knot 452 under the patient's skin 59 so as to place and tension the pledget 510 in abutting relationship with the outer fascia 57.

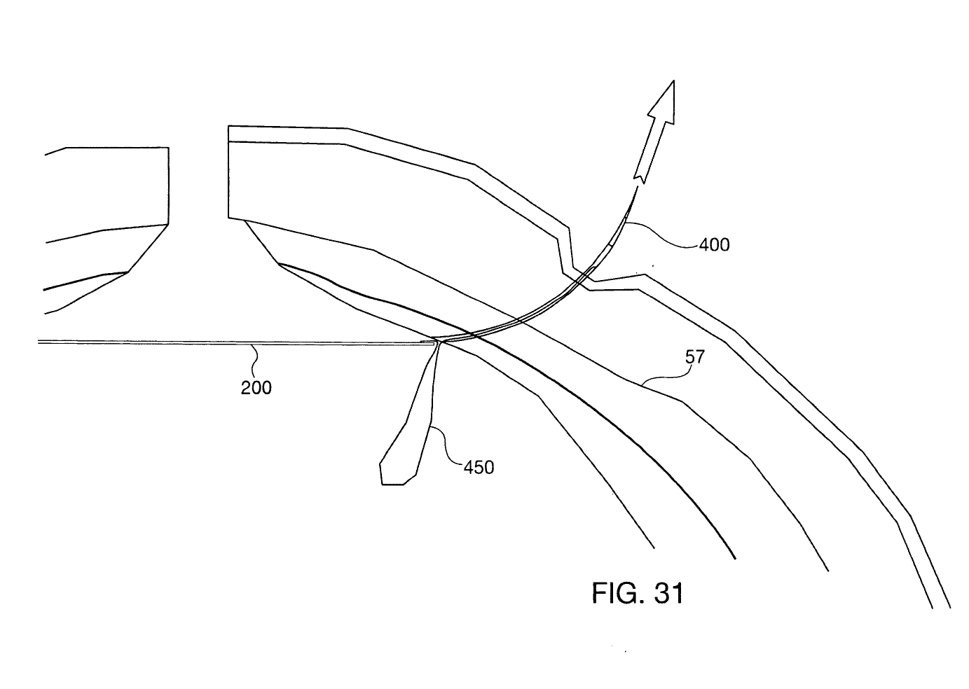

In FIGS. 31-33A the fixation fastener is a cleat 520 is shown schematically larger than normal scale for illustrative purposes as a tubular member for passage of one or more sutures 450 through its central bore. The cleat 520 has a pair staggered, radially opposed, axially oriented, slotted apertures 522 that have overlapping proximal portions oriented radially at approximately the midline of the cleat and distal portions terminating on opposite ends of the cleat. The staggered slotted apertures 522 construction enable passage of the suture 450 through the cleat 520 central bore. When the surgeon forms a knot 452 in the sutures 450 and advances the knot toward the patient's outer fascia 57 the cleat 520 is cammed in a position parallel to the outer fascia 57 surface and generally normal to the suture body axis, due to passage of the suture through the slotted apertures at opposite ends of the cleat.

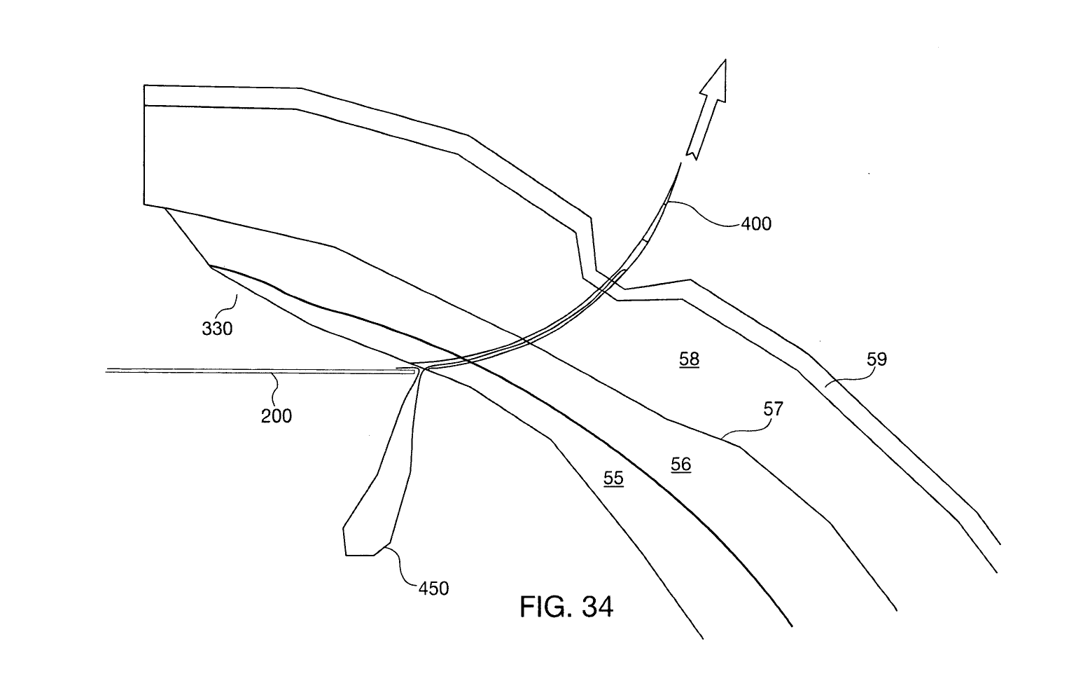

FIGS. 34-36 show an expanding anchor 530 fixation fastener, having a pair of radially opposed, axially oriented slots 532 formed in the midsection of the generally tubular body. The tubular body enables sliding passage of sutures 450. As the anchor 530 tubular structure is compressed from both ends, material forming both sides of the anchor slots 532 is bowed radially outwardly and forms a triangular truss 534.

An anchor delivery instrument 536 includes a trocar-like front collar 537 that has a central bore and full-length axial slot (not shown) that enables passage of a suture through the central bore. The suture may be released from the front collar 537 by radial translation out the peripheral full length slot. A tension wire 538 is attached to the collar 537 and allows retraction of both components from the patient's body by pulling the wire. A compression collar 539 has a central bore for passage of a suture.

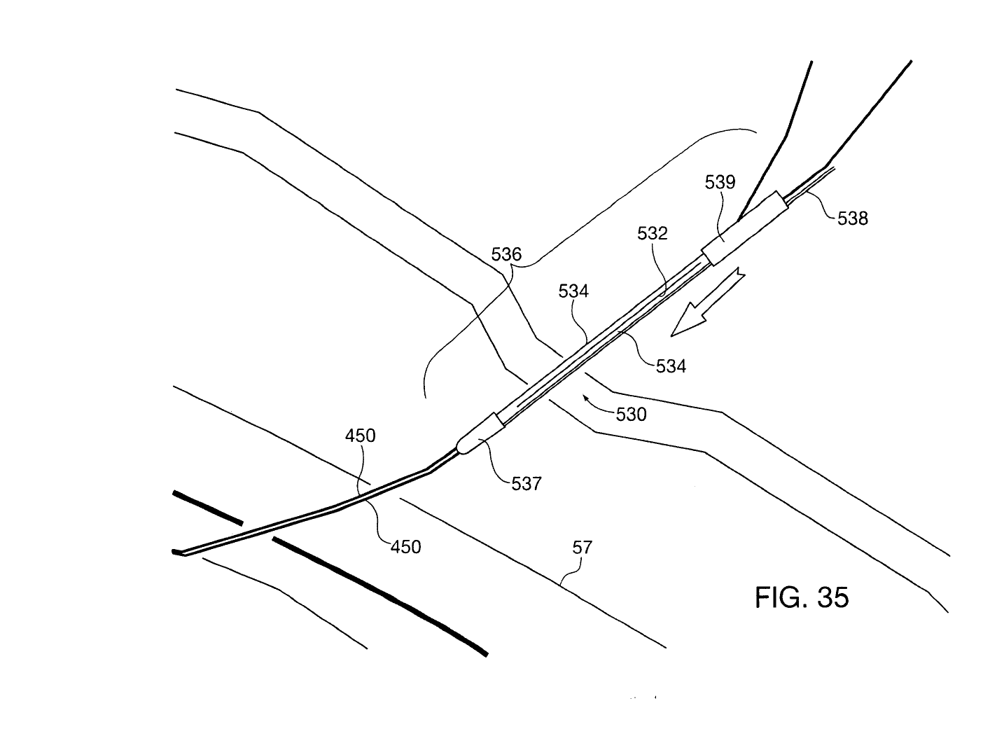

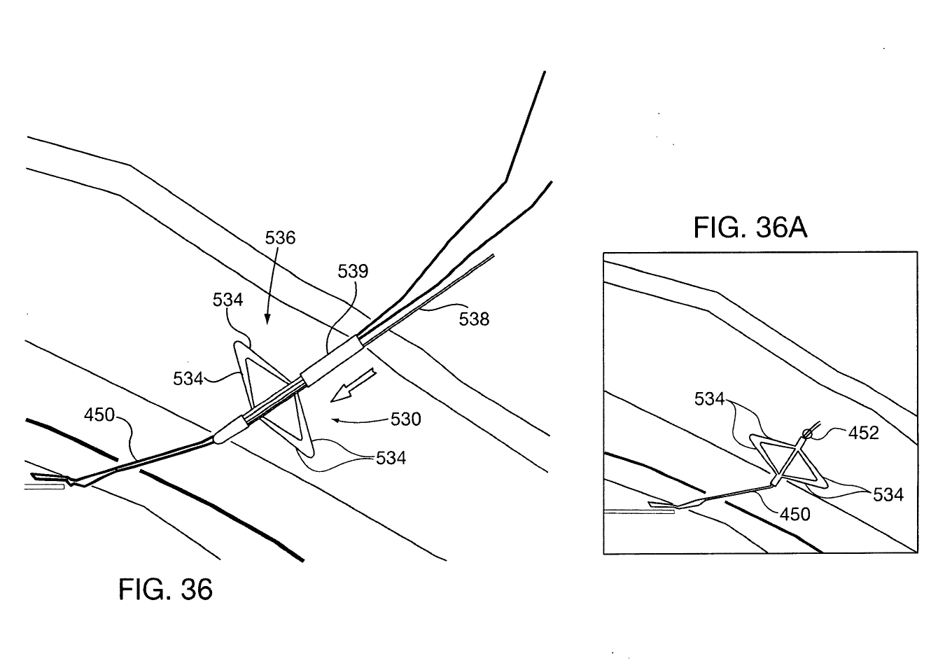

Referring to FIG. 34, a suture 450 is advanced from the patient's abdominal cavity through the repair mesh 200 and the abdominal wall tissue as has been performed with the other fixation fastener embodiments. The anchor delivery instrument 536 and anchor 534 are threaded on one or more legs of the suture 450 and they are advanced so that the front collar 537 is in proximity to the patient's outer fascia 57. Next, referring to FIGS. 35 and 36, the tension wire 538 is retracted while simultaneously advancing the compression collar, so that the anchor 534 is axially compressed, and thereby bows out the trusses 534 radially away from the suture 450. The front collar 537 is separated from the suture 450 by passing the suture through the full length axial slot formed therein. The anchor delivery instrument 536 components are retracted from the patient. Thereafter the surgeon forms and advances knot 452 in the suture 450, as described with respect to the other fixation embodiments, so that the suture is captured under desired tension between the repair mesh 200 and the anchor 530.

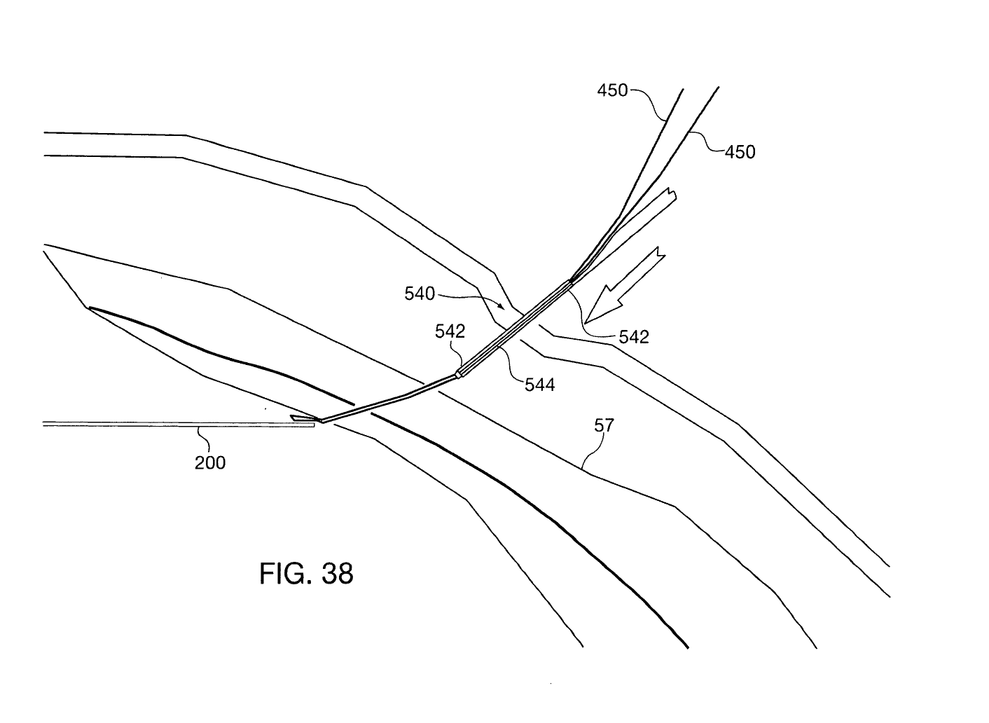

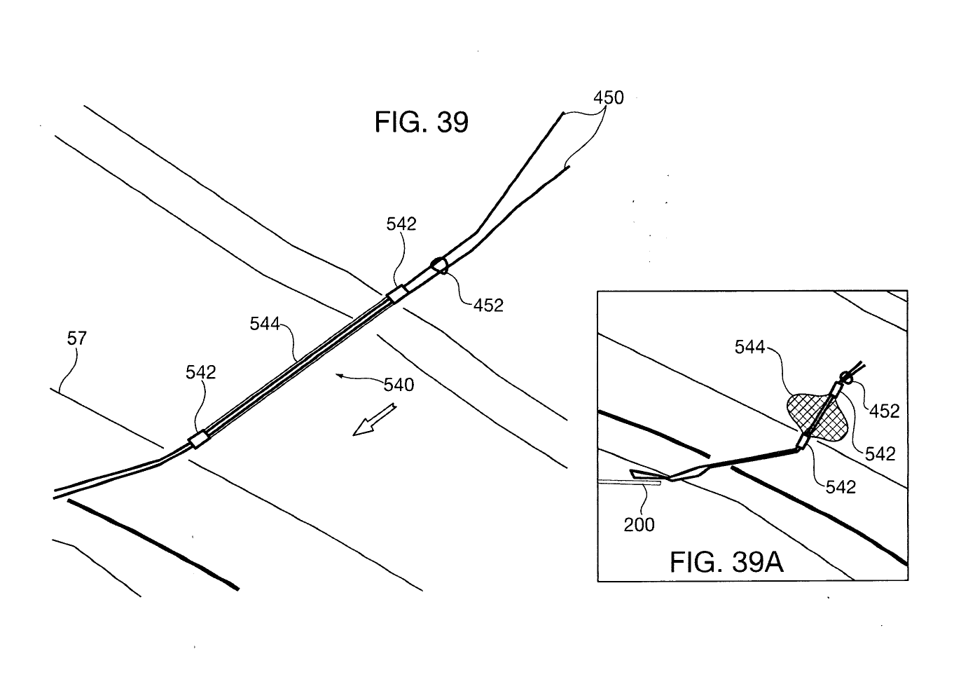

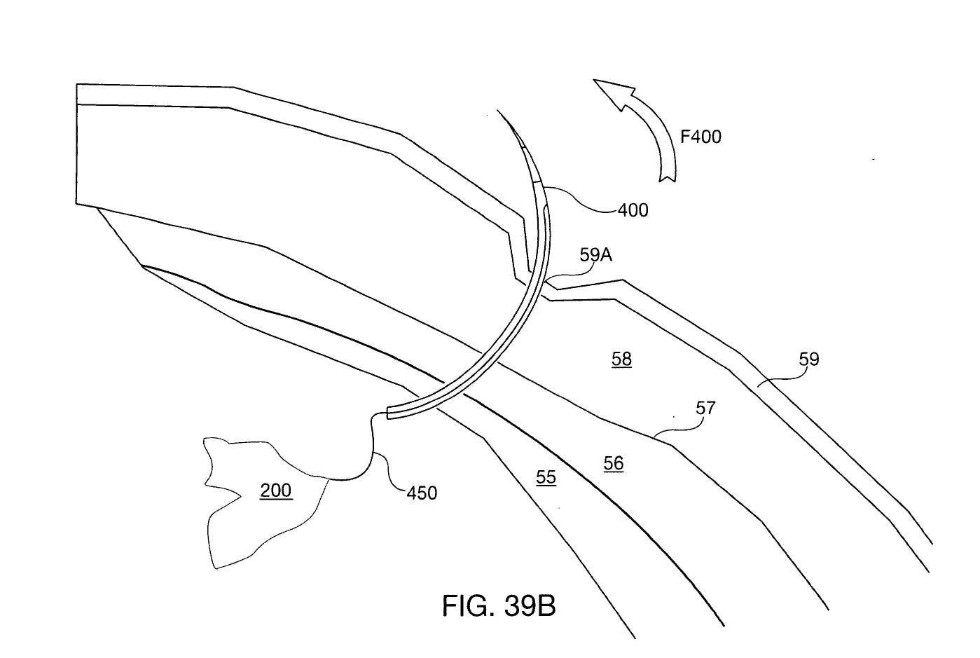

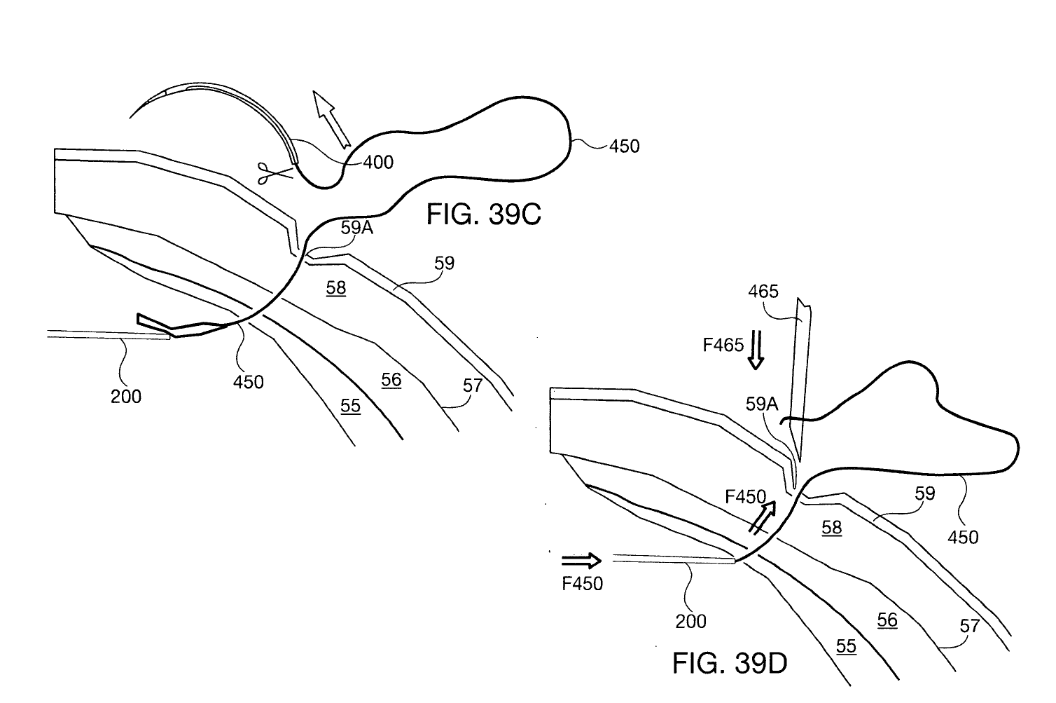

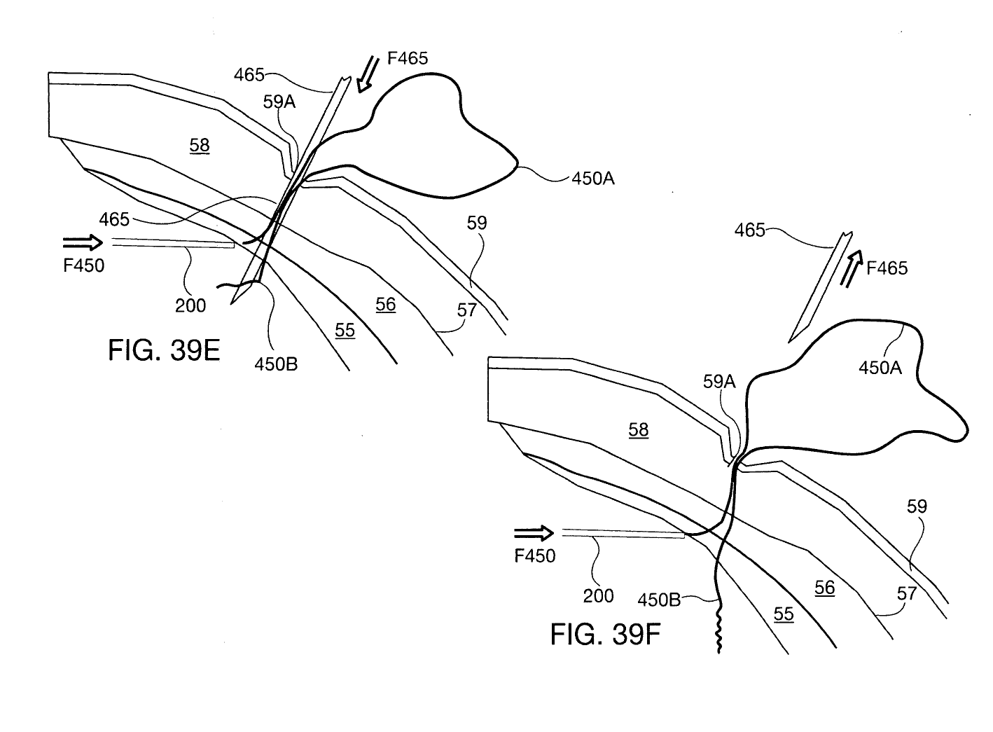

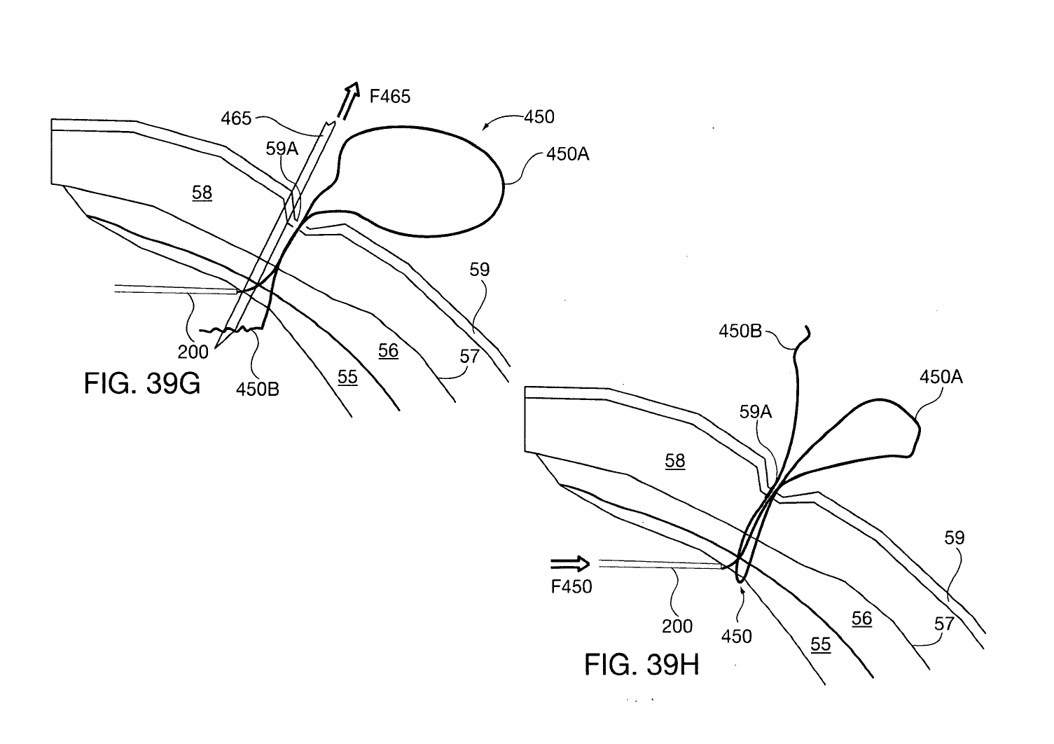

Another primary fixation fastener expanding braid anchor 540 is shown in FIGS. 38-39A. The general functional concepts of a radially outwardly bowed fastener and delivery instrument 536 that were employed in the anchor embodiment 530 of the immediately prior drawing descriptions are applicable to the braid anchor embodiment.

As described in connection with other fixation fastener embodiments, a needle and sutures 450 are advanced through the patient's abdominal tissue. The delivery instrument (not shown) captures braid fastener 540 between the front and compression collars.

Braid fastener 540 has a pair of braid tubes 542 that axially sandwich a length of coreless braided cable 544 between them. The braided cable 544 may be constructed of a polymer such as polyester or polypropylene. Relative compression of the pair of braid tubes 542 with the delivery instrument 536 in the manner described with respect to the expanding anchor fastener 530 radially bows out the braided cable 544. After retraction of the delivery instrument components, creation and advancement of knot 452 in the suture 450 captures the tensioned suture between the hernia mesh 200 and the braid fastener 540.

FIGS. 39B-39H show schematically primary fixation of the mesh 200 or other prosthetic device at a fixation site established by an instrument of the present invention with a common suture 450 alone and no other anchoring device. As shown in FIG. 39B, a device of the present invention advances a needle 400 attached to suture 450 with a constant radius needle pusher 440 or other needle advancement device compatible with the device of the present invention along a curved, retrograde path, from the patient's abdominal cavity peritoneum layer through the abdominal wall at a fixation site established by the instrument, and exiting the patient's skin 59 through a puncture 59A. The suture 450, as shown in FIG. 39C traces an inwardly curving, retrograde path created by advancement of the needle 400 through the patient's tissue. The needle 400 is passed through the patient's skin and is separated from suture 450, shown schematically by the scissors. The needle 400 is no longer needed for the remaining steps of this fixation procedure. If the needle is permanently affixed to the instrumentation as is some of the embodiments of the present invention that are described herein the suture is separated from the needle and the needle thereafter retracted back into the patient by the instrument.

Referring to schematic FIGS. 39D and 39E, retracting suture 450 (F450 schematic arrow) tensions the suture and causes the mesh 200 or other prosthetic device to abut firmly against the patient's tissue (here shown as the peritoneum 55). After initial suture 450 tension, it is reintroduced into the patient's tissue along a different path, preferably through or proximal the existing puncture 59A so as to minimize patient skin puncture site area, with a needle passer 465 or other device chosen by the surgeon. Reintroduction of the suture into the patient creates an anchoring loop bight 450A in the suture external the patient's skin. The needle passer 465 is advanced into patient's abdominal muscle along a path shown with force arrow F465.

Next as shown in FIG. 39F, the needle passer 465 is withdrawn from the patient through the puncture 59A, leaving behind the suture 450 anchored in tension along an inwardly curved path within the patient's abdominal muscle 56, the tension, shown as force arrow F450 maintaining tight abutment between the patient's inner abdominal wall peritoneum 55 and the prosthetic device mesh 200. The distal end of the suture 450B is left within the patient's body cavity temporarily, as shown in FIG. 39F.

Referring to FIG. 39G, the surgeon next reinserts the needle passer 465 through the patient's skin at a location proximal the original puncture 59A through a third needle pathway in the patient's tissue and picks up the suture's distal end 450B. As shown in FIG. 39H, the needle passer 465 is again retracted from the patient, force arrow F465, also retracting the distal end 450B of the suture back out of the patient's skin. The surgeon ties the suture distal end 450B and the loop 450A together (not shown) and may advance the knot formed therein under the patient's skin. As shown in FIG. 39H the suture 450 traces a serpentine path through three separate channels in the patient's tissue that is thereafter secured by a knot, affording secure fixation of the prosthetic device. The patient's dermal puncture site 59A zone is minimized by closely spacing needle entrance and exit sites.

It follows that the suture 450, or other linear fixation device with or without supplemental anchors, can be advanced and anchored within patient tissue, in order to retain a prosthetic device in firm abutment with the tissue, by inserting a needle or other device with the instruments of the present invention from patent interior to exterior, or, in reverse from the patient exterior to the interior. For example, if the device of the present invention inserts a needle from the patient exterior to the interior towards the prosthetic device once the needle is proximate the prosthetic device 200 the suture 450 can in turn be attached to the needle and the needle then retracted from the patient. The suture will now be exterior the patient tissue of interest and can be tensioned by the surgeon before re-inserting the suture end into the patient, with or without any of the primary fixation anchoring devices shown and described herein in FIGS. 25-39A or by the suture 450 return loop shown in FIGS. 39B-39F. No matter whether the fixation needles are advanced from inside/outside the patient or reverse, the instrumentation and methods of the present invention help the surgeon to establish prosthetic device fixation sites in the patient tissue automatically upon deployment of the instrument. In many of the embodiments described herein the fixation instruments and methods establish a plurality of prosthetic fixation sites simultaneously.

### Secondary Fixation

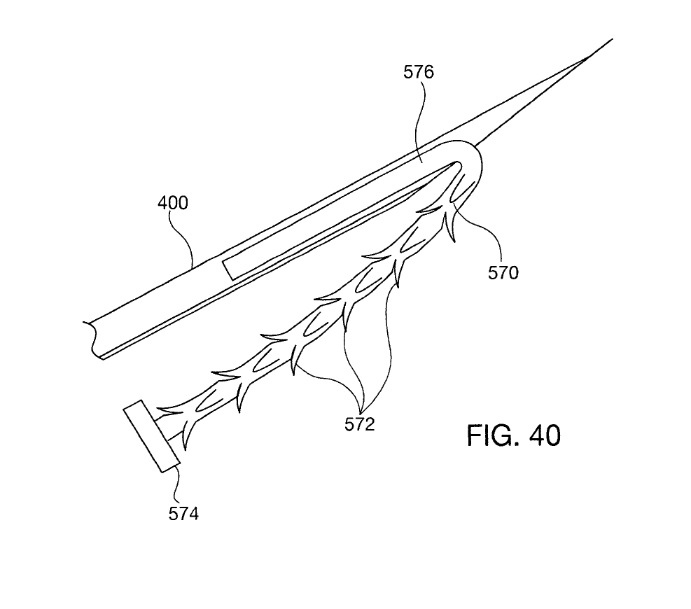

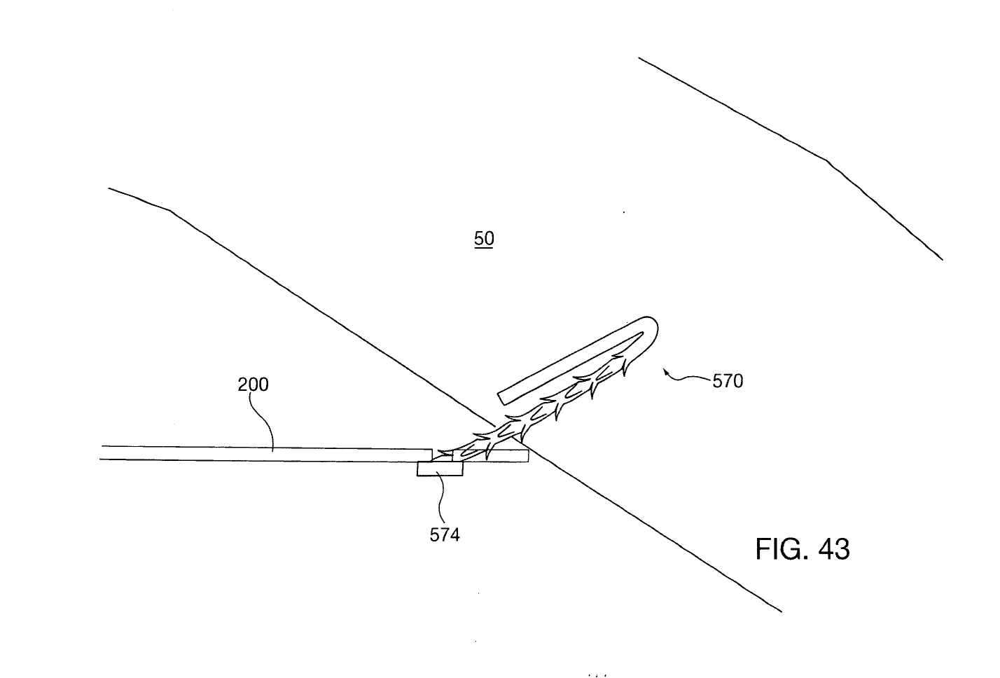

The surgeon may wish to affix the repair mesh to the patient in additional secondary locations beyond the primary fixation points created with the instruments and methods of the present invention. An additional aspect of the present invention is the ability to use barbed sutures 570 for secondary affixation of the hernia repair mesh, as shown in FIGS. 40-45. The barbed suture 570 has barbs to prevent the suture from backing out of tissue and a backstop 574 that may be constructed in accordance with the teachings of the primary fixation barbed suture 500 previously discussed herein. The suture 570′s non-barbed leader 576 is loaded into a hollow needle 400 for driving advancement into the patient from the abdominal cavity through the mesh and tissue. The needle 400 is advanced until the suture backstop 574 is snugly against the mesh 200 and thereafter retracted, leaving the suture 570 embedded in the patient's abdominal wall 50, where the suture barbs 572 engage surrounding tissue and prevent the suture from backing out. A fully deployed barbed suture 570 affixation anchor is shown in FIG. 43.

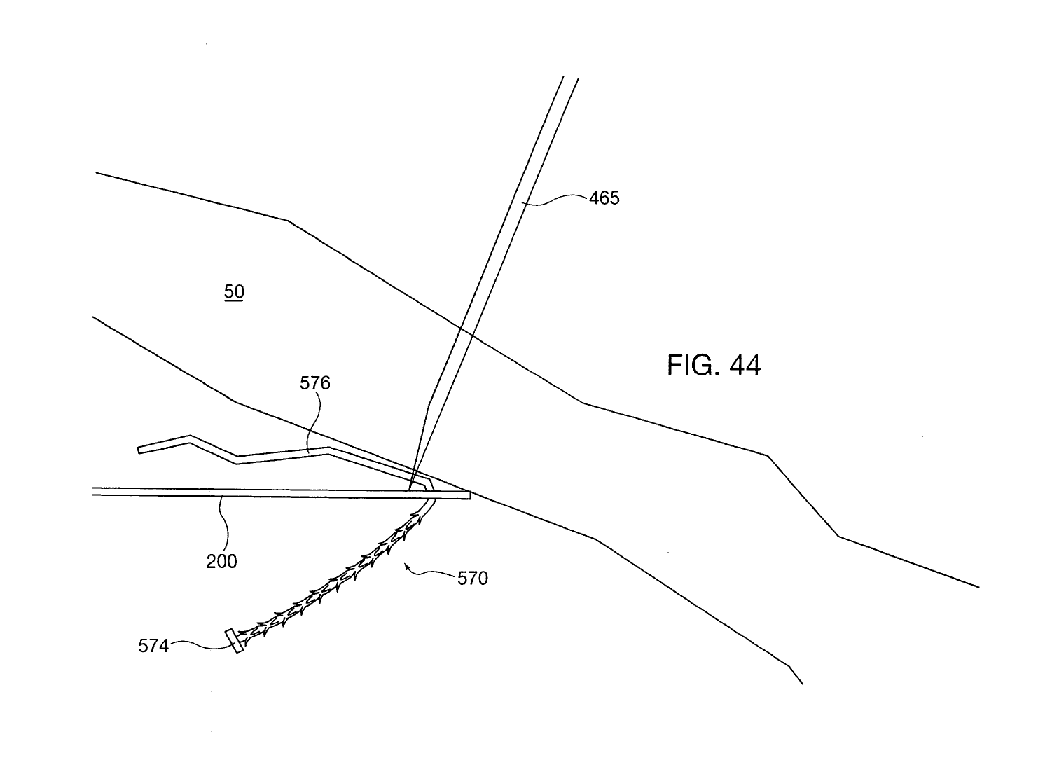

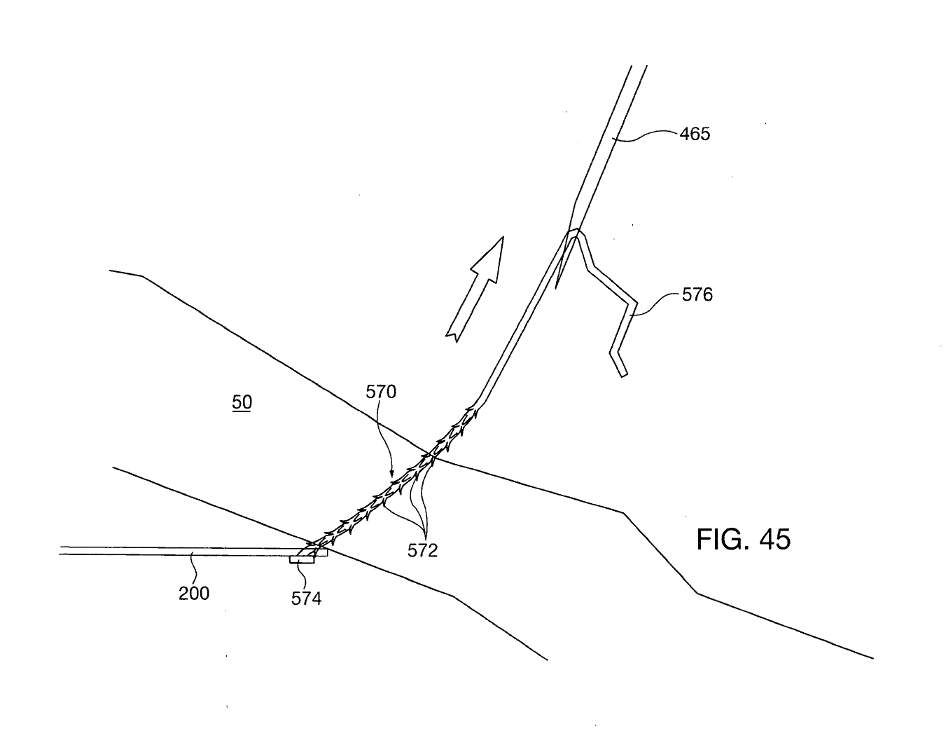

FIGS. 44 and 45 show an alternative method to affix the barbed suture 570 to a patient by pulling externally with a needle passer 465 rather than by pushing with a needle 400. In this embodiment, a needle passer is advanced through the patient into the abdominal cavity between the abdominal wall and the mesh 200. The needle passer 465 engages the suture leader 576. Subsequent retraction of the needle passer 465 tensions the suture 570 so that the mesh 200 is snugly abutted against the interior abdominal wall peritoneal layer 55. The tensioned suture 570 may be cut below the patient's skin.

### Umbrella Prosthetic Fixation Device

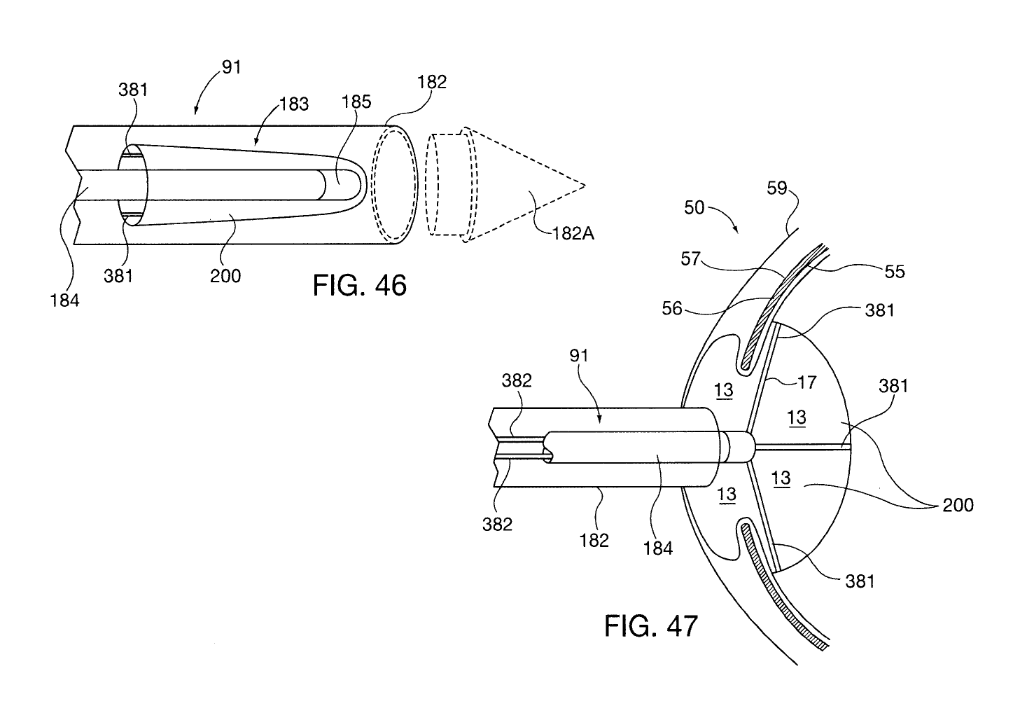

Other exemplary prosthetic fixation devices of the present invention are shown in FIGS. 46-49A. Application of these devices is described with respect to a ventral hernia repair.

First, a surgeon performs a standard laparoscopic adhesiolysis and reduction of a hernia defect preferably using only 5 mm or smaller trocars. While the inventive device allows for the use of 5 mm or smaller trocars, this portion of inventor approach can be done in any manner which the surgeon deems appropriate.

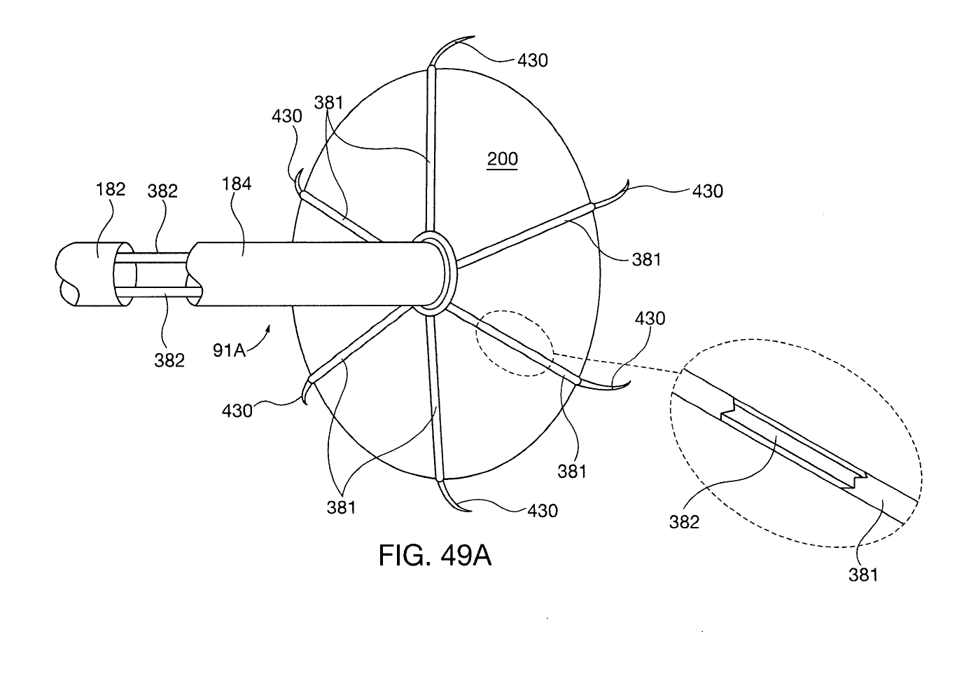

Then, following completion of dissection, mesh deployment device 91, shown in FIG. 46, is inserted, as shown in FIG. 47, through a separate umbrella cannula 182, similar to a trocar, in the center of hernia defect. Device 91 is formed of hollow rod 184 having mesh 200 collapsed around rod tip or end 185 and longitudinally and circumferentially extending around the rod and in a direction rearward of that end. The mesh is held in place by radial supporting struts (legs) 381 (only two of which are shown in FIG. 46) pivotally coupled to end 185 and spaced at approximate equal angular distances around end 185. Each such strut 381 is metallic or plastic, and has a hollow core. A sufficient strong and biocompatible pushrod 382, such as titanium, shown in FIG. 49, longitudinally extends through each strut 381 with, for simplicity, only two such struts and their corresponding pushrods 382 being illustratively shown (the remaining struts are identically configured).

The mesh 200 will be contained in canister or trocar 182 that has a sharp and appropriately shaped piercing tip 182A designed for ready entry into the peritoneal cavity through the center of the hernia defect. The tip 182A then opens or separates from end of the canister to allow the mesh 200 to enter into the peritoneal cavity. Once entry has been effected, the mesh 200 will then be opened, i.e., extended, to yield a structure similar to an opened umbrella. The canister 182 can be held in the body to help maintain pneumoperitonem pressure 55, and then removed once deployment is confirmed. Both the canister 182 and tip 182A will then be removed as they are no longer needed.

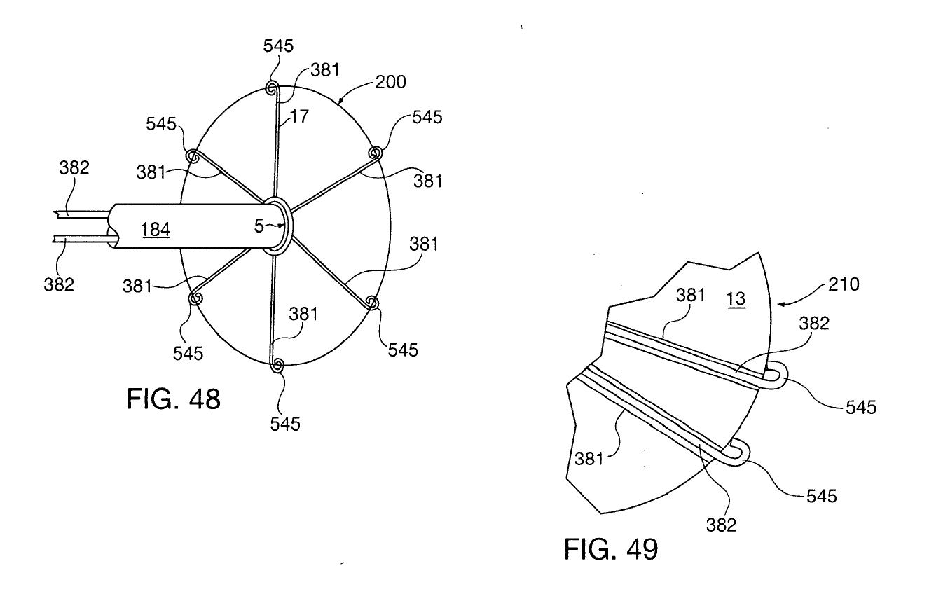

Mesh 200 is attached to a circularly-shaped underlying supporting lattice that is metallic or plastic, effectively formed by all of struts 381. This lattice is shown in FIG. 48 with all the struts 381 having been being fully extended into position, with all the struts effectively forming spokes, located at approximately equal angular distances, around a common underside of disk shaped mesh 200. Alternatively, the mesh 200 does not have to be affixed to the lattice structure struts 381 directly: it can be folded over the struts in loose, flaccid fashion and coupled to the struts by sutures passing through the struts as was shown in previously described embodiments of this invention. Indirect coupling attachment of the mesh 200 to the struts 381 via flexible sutures functions similarly to the parachute-type mesh deployment described previously in this application.

Pushrod 382, which is situated within and longitudinally extends through each strut 381, has hook shaped NITINOL® metal anchoring device 545 situated and engaged with a proximate end of that pushrod. The device 545 is capable of being rotated through 300 degrees, if not more, by suitable rotation of its rod 382. Each of the pushrods 382 then longitudinally extends not only through its corresponding strut 381, but also, along with all other such respective pushrods for their struts, through a hollow longitudinal core of hollow rod 184 and for a sufficient distance thereafter to enable a surgeon to manipulate a distal end of each of the rods 382. Anchoring device 545, typically a clip, is attached to a corresponding point along outer peripheral edge 210 of mesh 200.

Once the mesh 200 is suitably opened, such as by the surgeon suitably pulling a distal end of each one of pushrods 382, to create movement in a direction opposite to that shown by arrow F in FIG. 49, and hernia defect is covered by the mesh to an adequate margin, each of the NITINOL® metal anchoring devices 545 will then be anchored into proper position in the posterior abdominal wall. That position is typically several centimeters from a corresponding edge of the hernia defect. This occurs by the surgeon either pushing, in the direction shown by arrow F in FIG. 49, and/or rotating, like a corkscrew, or otherwise manipulating the distal end of the corresponding pushrod 382. Doing so pushes the anchoring device 545, which is already secured to a corresponding point on the mesh 200, into a corresponding proximate point on the abdominal wall, thus driving through an adjacent fascia.

After the surgeon has confirmed proper placement of each of anchoring devices 545, an energy source is then applied to each of these devices, with that source being, e.g., kinetic mechanical, thermal or light energy, and directed to each junction of a pushrod 382 with its corresponding NITINOL® metal anchoring device 545. Doing so allows the pushrod 382 to separate from its anchoring device 545 and that device to fold (snap) into place, i.e., loop back onto itself as an inwardly directed curve as is functionally performed by the suture 450 orientation in FIGS. 39B-39F. Thus the anchoring device 545 effectively changes its shape from a hook to a substantially, if not fully, closed ring, thus properly and securely affixing the mesh 200, collectively at points along its periphery, to the abdominal wall in situ. The canister 91 and the lattice of the hollow rod 184, radial supporting struts 381 and pushrods 382 will then be removed, thus leaving only the mesh 200 and NITINOL®, metal anchoring devices 545 within the body.

Though not specifically shown, each anchoring device 545 may be maintained in a suitable sheath, e.g., a plastic casing, prior to its use. Through suitable manipulation of the distal end of its pushrod 382, the surgeon can first position the device 545 proximate to its final installed position in the abdominal wall, then through further manipulation, release the device from its sheath and, through a last such manipulation, secure the device in position as an anchor by suitably driving it through the fascia. Then, through application of suitable energy, as discussed above, the device will snap back on itself and thus secure the mesh to the fascia. Using such a sheath will keep the device in a proper alignment through its entire installation, thus simplifying, facilitating, and expediting its proper installation.

Alternatively, the struts 381, which once deployed form spokes, containing the deployment rods 382 need not detach from the mesh, but instead, can stay attached to the mesh by glue, stitch or any other well-known suitable mechanism. Moreover, the device 91 can have a center hub (not shown) situated proximate to end 185. This hub can be either detached or left in situ after fixation is applied. During deployment, struts 381 are slid through the hub, similar to the lever arm cannula embodiment 170 of FIGS. 18-20. Both the hub and each strut 381 have complementary and mating detents. Once each strut 381 is fully extended, the detents on each engage, thus locking each strut into its maximum extension and also forming a pivot point between the strut and hub, thus allowing the strut to rotate radially outward into position from the hub. The device 91 also has a fixed ring type hook (anchoring device) 545 which is contained entirely within each strut 381 and upon application of kinetic energy (e.g., twist or push) would deploy outward and complete either a full or near full circle to secure itself and the mesh to the abdominal wall. Here, the hooks 545 may be made of materials other than NITINOL®, e.g., preformed metal hooks with spring memory. Further, pushrods 382 may not be necessary. Since both the hooks 545 and the struts 382 remain permanently attached to the mesh 200 in this embodiment, this provides added torsional and radial rigidity.

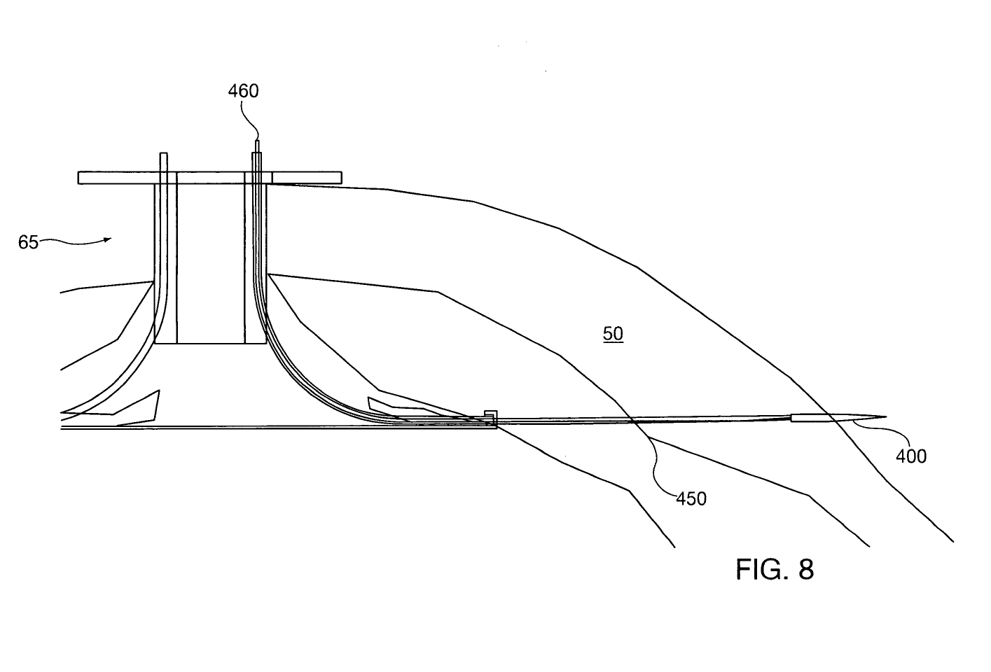

Moreover (also though not shown), rod 184 may be solid with longitudinally oriented channels positioned around its external periphery, each of which would serve as a guide for a corresponding one of struts 381 (functionally similar in operation as the L shaped cannula 65 embodiment of FIGS. 7 and 8). The distal end of the struts 381 could be pivotally secured to a central hub which slidably moves longitudinally along the peripheral outer surface of rod 184 in the direction of end 185 to fully deploy the struts 381 and to open and straighten the mesh 200 in much the same fashion as an umbrella is opened and to form a similar arrangement of spokes. Once the hub is locked flush to the mesh, the final travel distance would push the NITINOL® metal anchoring devices out of their respective sheaths. In this instance, the purpose for pushrods 382 would be to deploy and secure anchoring devices 545 into their proper positions. Here too, if fixed position anchoring devices are used, there may be no need for pushrods 382. Further and readily apparent to anyone skilled in the art, two hubs may be used. Here, a first hub is attached to mesh 200 and connected to all struts 381. A second hub abuts against the first hub and would deploy the mesh and lattice by pushing the first hub slidably down rod 184 to extend the struts—again much like an umbrella. In this case, the first hub then locks into position when the struts 381 are extended, and then allows the pushrods 382 to be deployed to position and secure the anchoring devices 545 into position. The second hub, like the cannula 182, would be removed from the patient after proper deployment of the struts 381 and mesh 200.

Struts 381 are attached to mesh 200 with either suture thread, which can be released, or a pivot joint which also can be released.

FIG. 49A shows an umbrella-type fixation device 91A that can be introduced to the herniation site from within the patient's abdominal cavity through existing known lateral laparoscopic surgical techniques. In this embodiment curved memory metal needles 430, that may be constructed of NITINOL® alloy, are folded back into the umbrella cannula 182 prior to deployment. When the rod 184 is advanced out the cannula 182 the needles 430 assume their relaxed configuration as a bowed, convex dome. The needles 40 are advanced into the patient's tissue by advancement of the pushrods 382 by surgeon through a laparoscope (not shown).

Meshes 200 of different sizes, such as of differing diameters, can be used to cover differently sized hernias. Moreover, existing mesh products designed for hernia repair can also be used. During surgery, the surgeon can choose the appropriately sized mesh 200 and insert it, along with its underlying supporting lattice 184, 382 into a canister 182 for subsequent deployment. Alternatively and to simplify deployment, at its time of manufacture, the mesh 200 and its lattice 184, 382 can be pre-installed into a corresponding canister 91 and supplied, in sterile packaging, as an integral unit to, e.g., a hospital for subsequent use by a surgeon. Further, the mesh 200 need not be limited to being circular in shape (as shown in the figures), as the shape can be readily changed, if needed, to suitably accommodate the geometry of the hernia or other defect to be covered. In such a case, anchoring devices 545 would still be affixed to the mesh and along its peripheral edge. Since the mesh is contained in a canister and is sterile prior to its deployment, no increased risk of infection results from using differently sized meshes.

Through use of the anchoring devices, the mesh, or other prosthetic device, can be attached to polyester, polypropylene, PTFE, dermis, collagen, or any other tissue substitute that may be used by the surgeon to bridge a gap in the abdominal wall.

### Prosthetic Fixation Device Integrated System

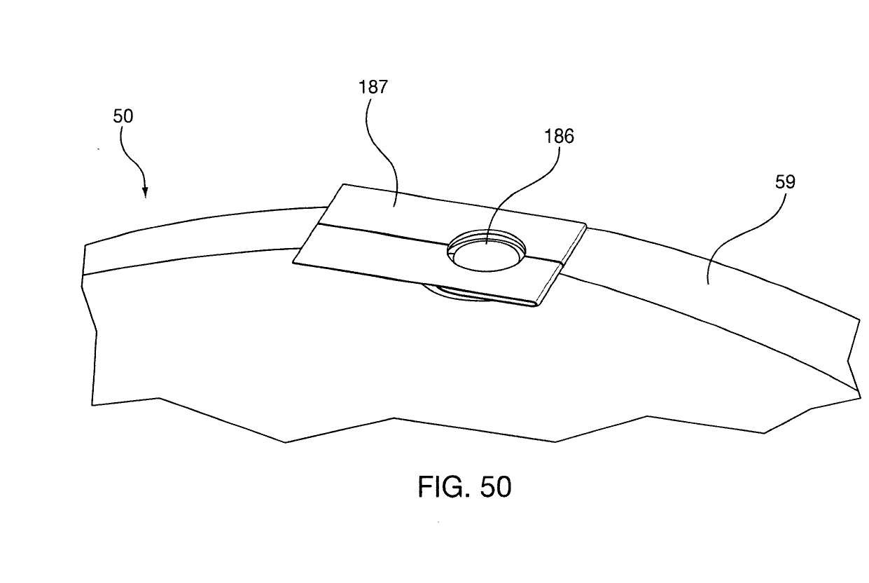

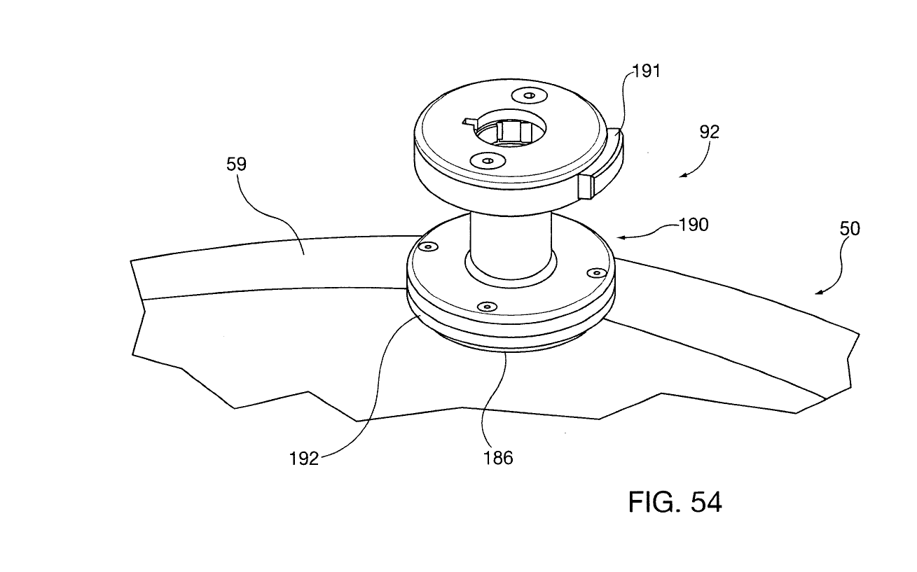

FIGS. 50-62 show an exemplary integrated system 92 for fixing prosthetic devices to patient tissue. As with other invention embodiments previously discussed, application of the integrated system 92 will be described with respect to repair of a ventral hernia by affixation of a mesh 200 to a patient's interior abdominal wall within the abdominal cavity, it being understood that the system can be applied to affixation of other types of prosthetic devices to body tissue.

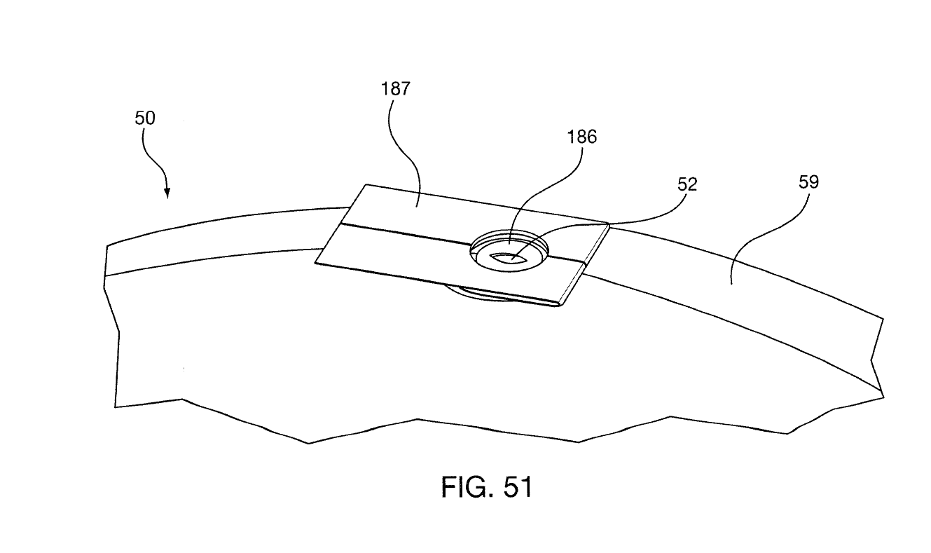

In FIG. 50 the patient has a ventral hernia within the abdominopelvic wall 50. The patient is prepared for surgery, using known surgical procedures; including insertion of one or more laparoscopes, not shown, and inflation of the abdominal cavity so as to create space between the patient's abdominal wall and viscera. A double sided adhesive ring 186 is affixed to the patient's skin, centered over the hernia defect. The exterior face of the adhesive ring 186 is covered with a peel away film 187. The surgeon creates a tissue incision 52 in the center of the adhesive ring 186, as shown in FIG. 51.

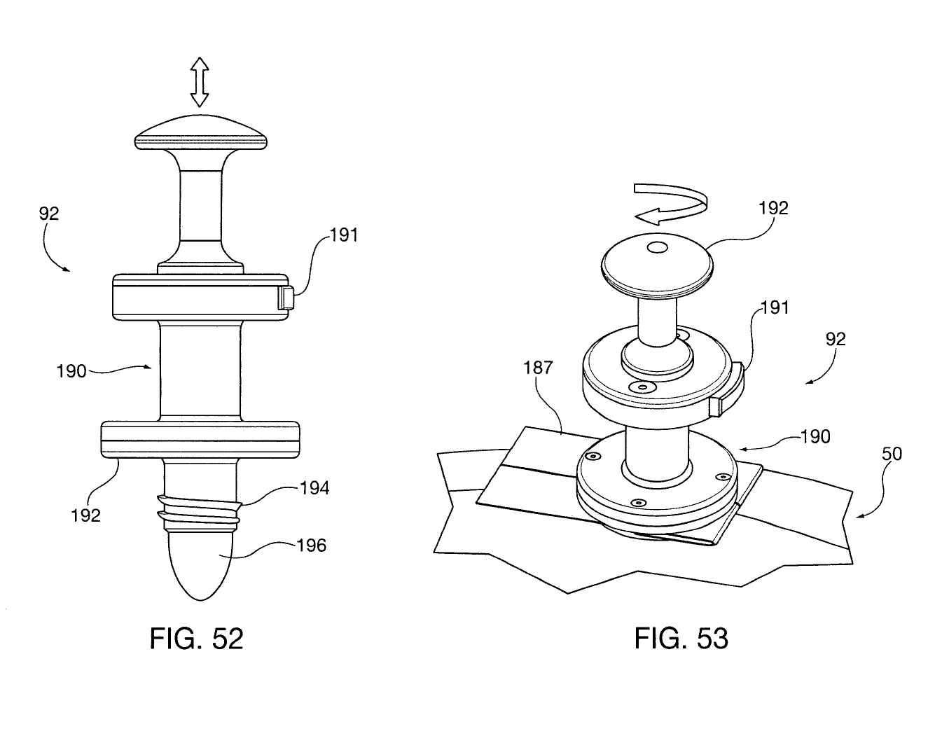

Referring to FIGS. 52-54, the integrated system 92 includes a port 190 with a spring-loaded axial lock mechanism 191 that enables the operating surgeon to lock other instruments into the port's central bore at a selected axial insertion position. The port 190 has a port collar 192 that in operation abuts against the patient's abdominal wall (or other selected body tissue) and advancing threads 194 to assist retention of the port in a fixed position within the patient's incision 52. The port 190 is inserted into the patient's incision 52 with the blunt tip obturator 196, inserted into the port central bore. The surgeon advances and twists the combined port 190 and obturator 196 into the patient incision 52 until the collar 192 contacts the peel-away film 187, after which, the film is removed, exposing the upper adhesive surface of the adhesive ring 186 so that it adheres to the port collar. Adhesive sealing of the skin surface 59, adhesive ring 186 and port 190 assists in maintaining positive pressure within the patient's abdominal cavity. Thereafter the obturator 196 is removed from the port 190 by release of the axial lock mechanism 191, allowing its replacement with the delivery device 197.

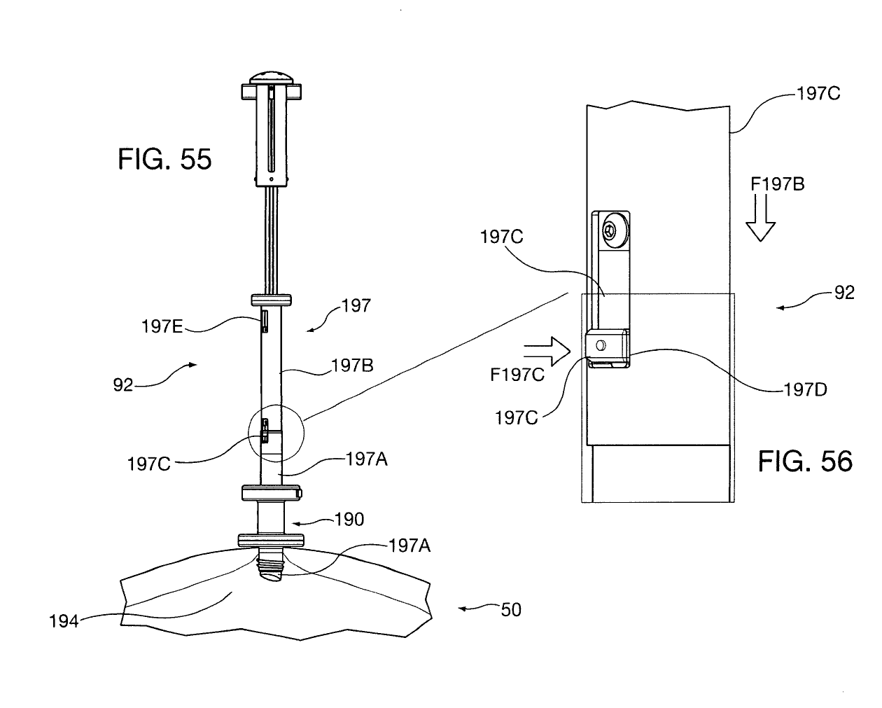

Referring to FIGS. 55-57, the delivery device 197 includes a mesh applicator 197A cannula portion that is received within the port 190 central bore and is locked into a selective position with the axial lock mechanism 191. Generally the surgeon inserts the mesh applicator 197A into the port 190 so that its tip is visibly projecting from the port advancing threads 194 when viewed through a laparoscope within the patient's abdominal cavity. The delivery device 197 includes a mesh deployment plunger 197B retained within the mesh applicator 197A in reciprocable, telescoping fashion. Mesh deployment plunger 197B may include a compartment for storing a furled mesh (not shown in stored position) or other prosthetic device. Pushing plunger 197B (see schematic force Arrow F197B) into the patient's body, after disengaging the mesh deployment plunger detent 197C from a detent engagement aperture 197D defined by the mesh applicator cannula tube 197A (see schematic force arrow F 197B) deploys furled mesh 200 and its attached fixation sutures 450 into the patient's abdominal cavity. Alternatively in lieu of mesh deployment with the delivery device 197, the surgeon may manually insert the mesh 200 into the patient's abdominal cavity through the incision 52 with any other chosen instrument, or laterally with a laparoscope.

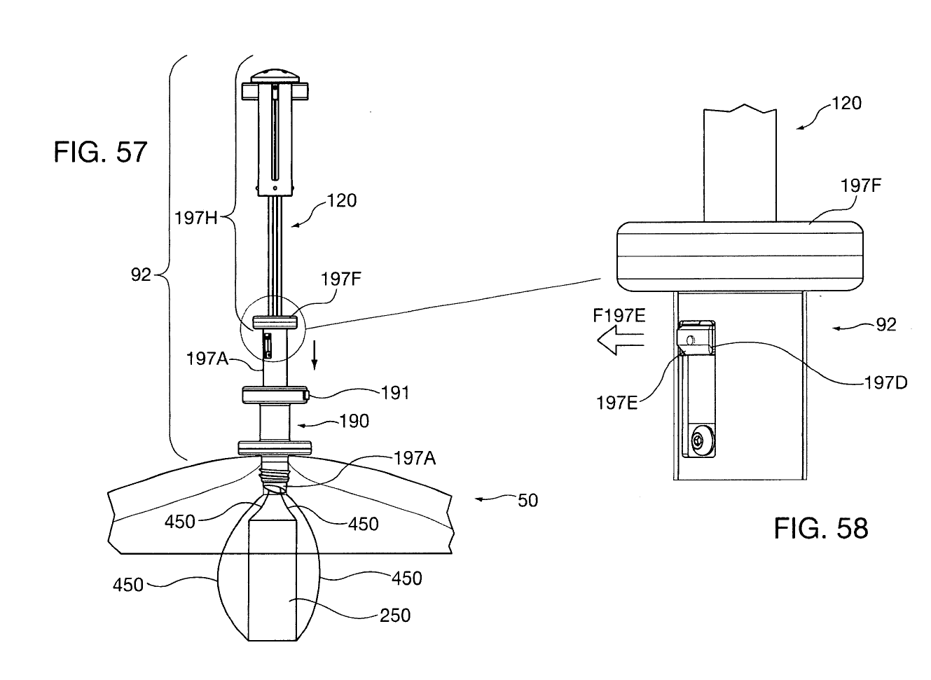

After mesh 200 deployment, the mesh deployment plunger 197B is telescoped into the mesh applicator 197A, as shown in FIGS. 57 and 58. The surgeon confirms that that the plunger assembly detent 197E (force arrow 197E) of collar 197F is locked into the detent engagement aperture 197D of the mesh applicator 197, so that the plunger assembly 197H may be actuated. The plunger assembly 197H operates similar to the embodiment shown in FIGS. 1A-4 previously described, and includes needle advancement channels 120, plunger 130 and needle sliders 140.

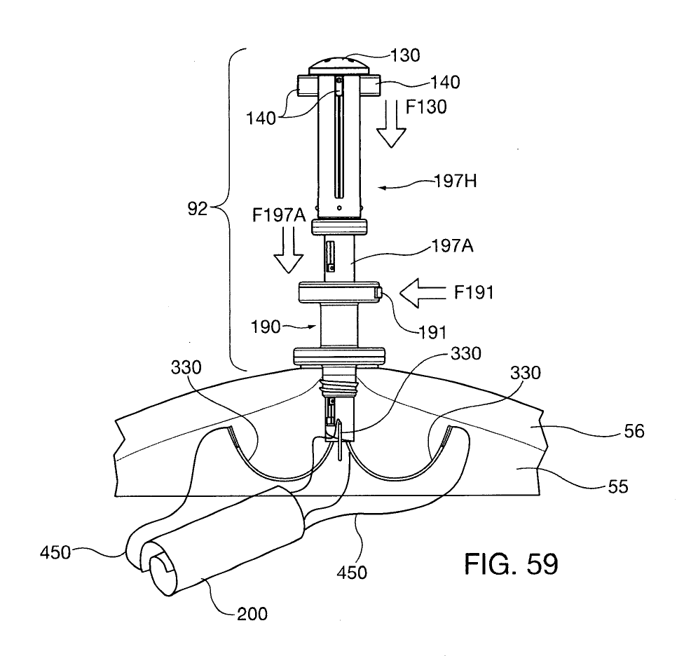

The deployment device 197 establishes simultaneously multiple fixation points/sites for the mesh 200 at the patient's intended prosthesis fixation site. Referring to FIG. 59, the surgeon releases the spring-loaded axial lock mechanism 191, as shown with the schematic force F191. Then, the surgeon advances the mesh applicator 197A deeper into the patient's abdominal cavity, in order to allow sufficient clearance to deploy the struts 330, as shown with the schematic force arrow F197A, and re-locks lock mechanism 191. As previously noted, maintenance of a pressurized abdominal cavity creates a void between the patient's abdominal wall and viscera.

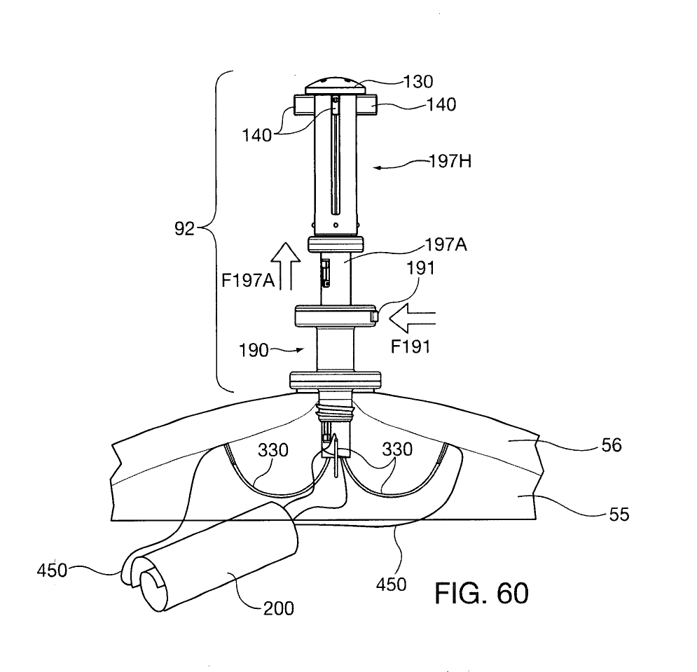

Depression of plunger 130 in the direction of schematic force arrow F130 (FIG. 59) advances the memory metal, hollow cannula struts/legs 330, allowing them to assume their pre-curved relaxed state. The strut 330 tips translate a retrograde path from pre-deployment state before depressing the plunger 130 (i.e., the tips point into the patient's abdominal cavity in the original direction of the applicator 197 advancement into the patient), to where they point in the generally opposite direction greater than normal to the applicator 197 initial advancement direction (i.e., the tips now point laterally/backward toward the patient's abdominal wall). Note that the strut 330 curvature preferably also traces an inwardly-recurving profile: i.e., initially the strut curves radially away from the centerline of the device 197, but the distal tip portion re-curves radially inwardly toward the device 197 centerline. As shown in FIG. 59, distal ends of sutures 450 are captured within the struts 330. The surgeon next unlocks the axial collar 191 (force arrow F191) and retracts the mesh applicator 197A (force arrow F197A in FIG. 60) until the distal end tips of the struts 330 are oriented into abutting contact with the patient's abdominal wall tissue (here shown the peritoneum layer 55). Now the surgeon has established a multitude (here four) primary fixation contact points/sites for the prosthetic mesh 200. As the mesh 200 suture 450 is now coupled to a strut 330 at each respective fixation site, the mesh 200 may now be selectively reoriented precisely at each fixation site by maneuvering the suture.

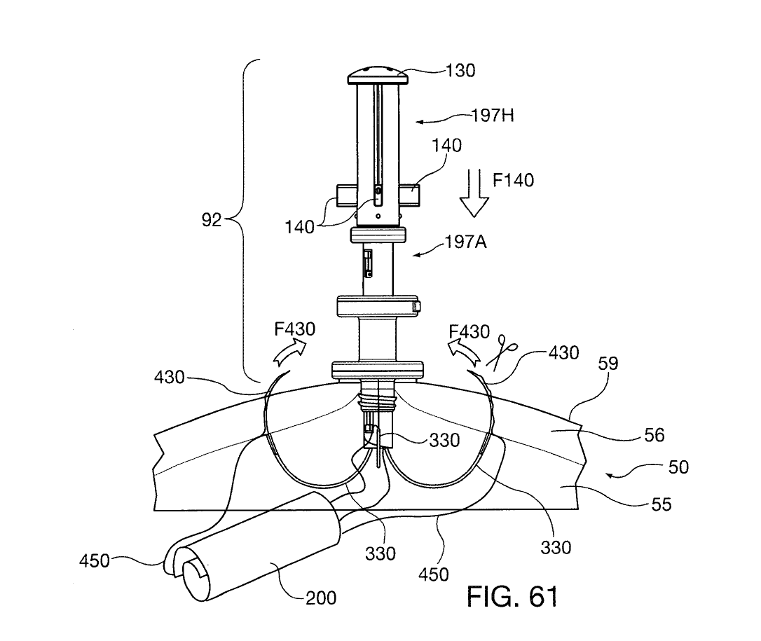

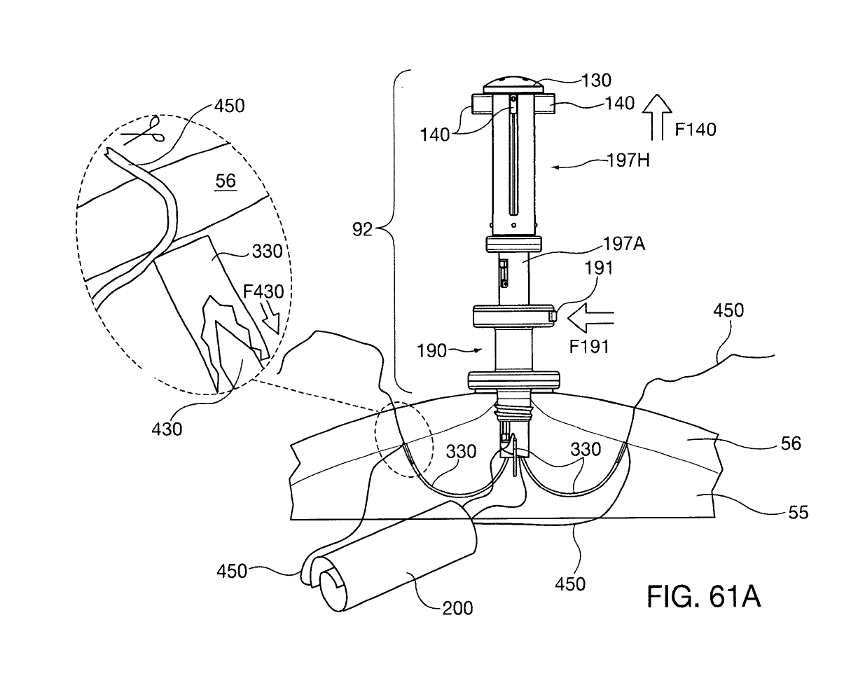

Once the fixation sites are established by abutting contact of the strut 330 tips and the patient tissue, the surgeon advances serially each of the four needle sliders 140 in the direction of the force arrow F140 shown in FIG. 61, so that the pre-curved needles 430, that are affixed to the plunger assembly 197H, advance the full thickness of the patient tissue and exit the skin 59. Note that the combined action of the pre-curved struts 330 and the pre-curved needles 430 cause the suture 450 to trace an inwardly curving, retrograde path through the patient's tissue. Upon advancement of all of the needle sliders 140 all of the mesh 200 fixation sutures 450 are affixed within the patient's tissue at the pre-selected fixation points/sites established by the struts 330 tips. Suture 450 tails now exit the patient's skin for each corresponding fixation site. As shown schematically by scissors in FIGS. 61 and 61A, the surgeon separates the suture 450 tails from the needles 430.

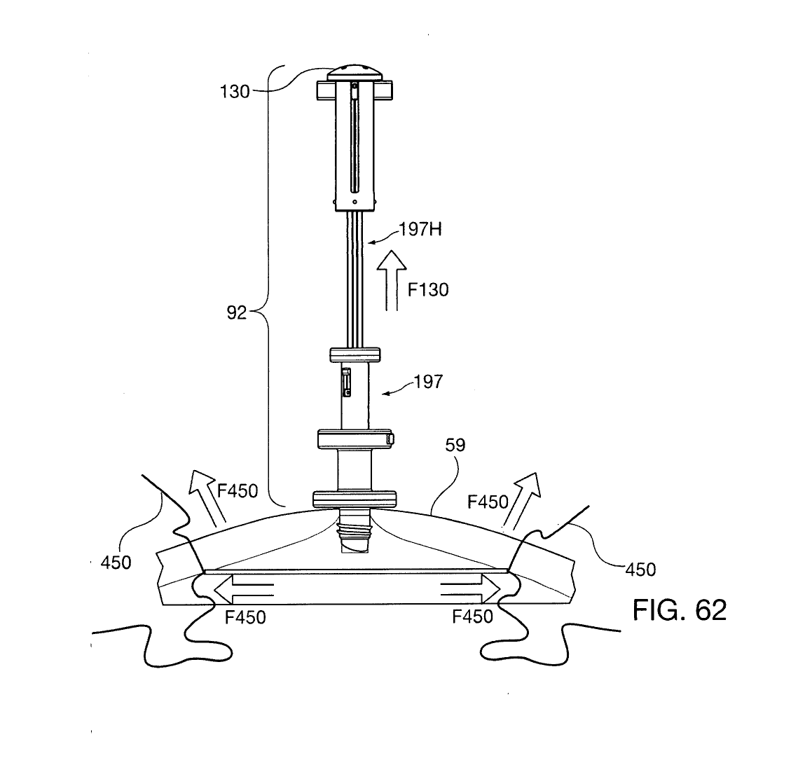

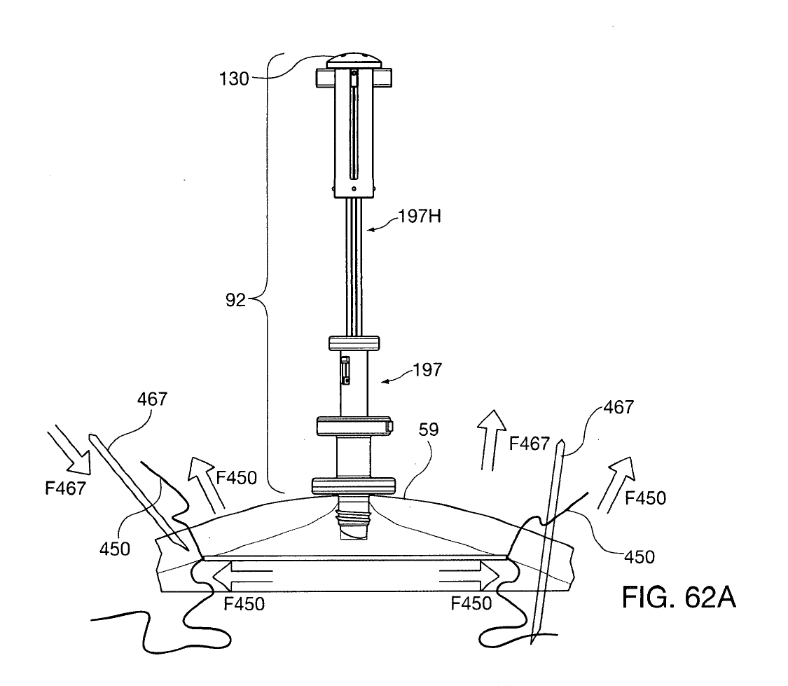

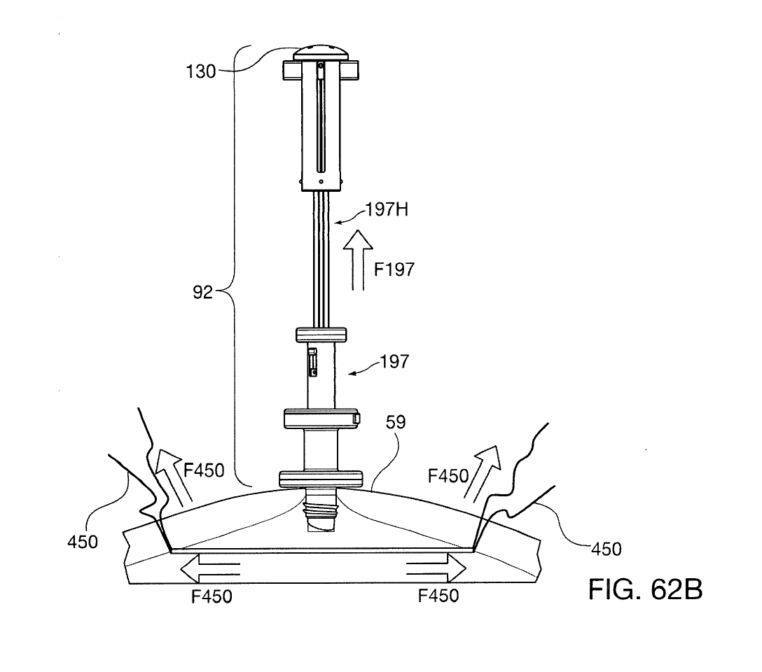

After suture 450 separation, from the needles 430, the surgeon retracts the needle sliders 140, shown by schematic force arrow F140 in FIG. 61A, so that the needles 430 return back to their retracted position captured by the struts 330. Once the needles 430 are retracted, the surgeon retracts plunger assembly 197H as shown with the schematic force arrow F130 in FIG. 62, thereby retracting the memory metal struts 330 and the needles 430 back into the instrument 197.

Thereafter the surgeon retracts and tensions the sutures 450 by pulling them, as shown with schematic force arrows F450 in FIG. 62. Tensioning the sutures 450 maneuvers them relative to the fixation sites previously established by the struts 330, and tensions the prosthetic mesh 200 about its periphery. This assures a firm, tensioned abutting contact orientation between the mesh and the patient's peritoneum layer 55 at each fixation point.

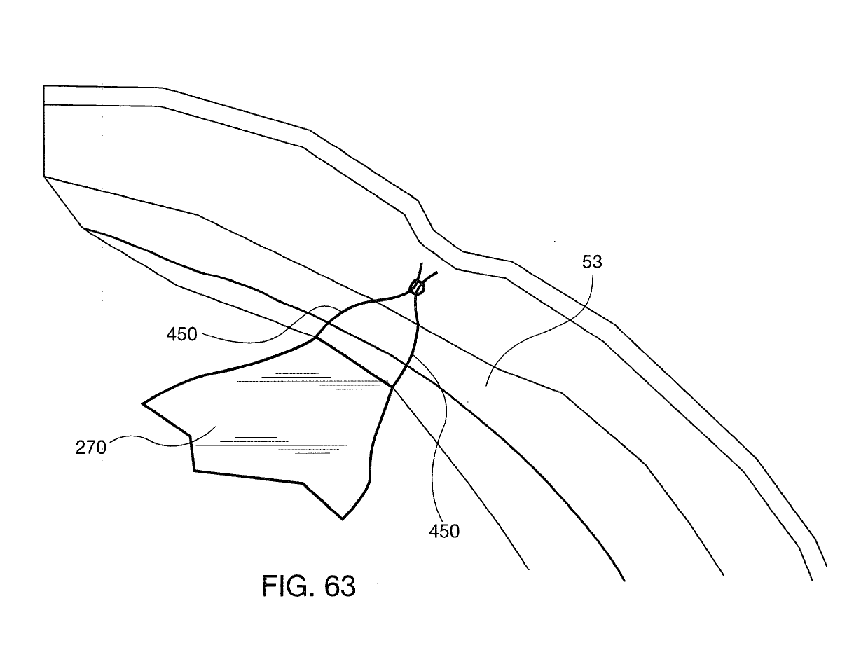

Referring now to FIGS. 62A and 62B, the surgeon may choose to use a double-suture 450 as a primary fixation device, if a double suture is coupled to the prosthetic device. The surgeon advances a needle passer 467 through the patient's skin 59 and other tissue through a different pathway than that formed previously by the needles 430. If desired, the needle passer 467 entry point may be proximal the exit point for the corresponding needle 430, but it is not required. The needle passer 467 picks up the loose suture 450 tail within the patient's body cavity and retracts it through the patient tissue where it exits the patient's skin. The two suture 450 tails may be affixed to each other to form a sub dermal knot as shown in FIG. 63.

The surgeon may anchor the primary fixation sutures 450 with any of the anchoring devices previously described, including the inwardly directed curving suture path shown in FIGS. 39B-39F. If desired, secondary fixation devices, such as shown in FIGS. 40-45 may be utilized to affix mesh 200 to the patient's tissue between the primary fixation sites 330.

### Alternative Fixation Device Configurations

While previous descriptions of fixation device applications have focused on ventral hernia repair, the present invention can be applied to alternative applications. In FIG. 63 a fixation device of the present invention was used to affix a generic prosthetic device]270 to patient tissue 53 by way of sutures 450 that were inserted into the tissue in an inwardly-curving, two dimensional spiral path.

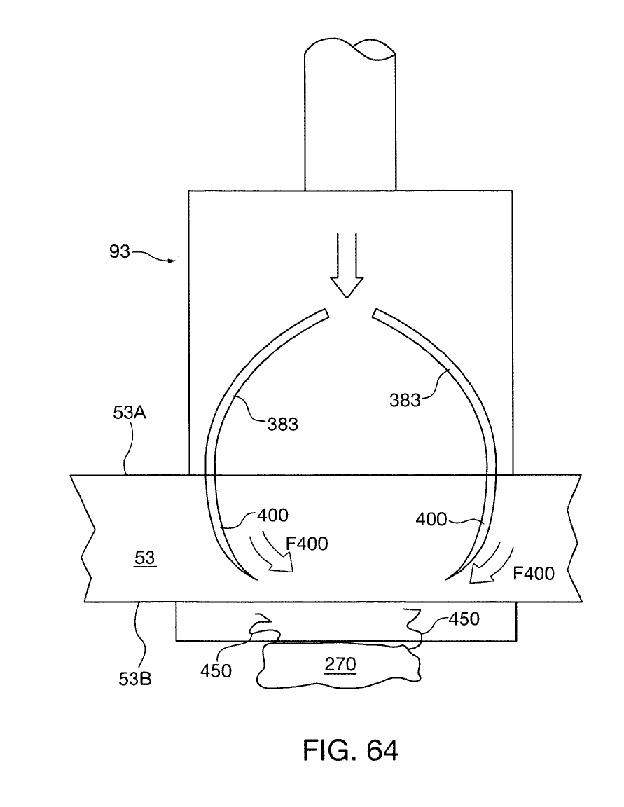

It is possible to configure fixation devices so that they can establish primary prosthetic fixation points from both outside and inside the patient. As shown schematically in FIG. 64 external fixation device 93 simultaneously establishes multiple fixation points with struts 383 abutting an external surface 53A of patient tissue, and causes fixation at those selected points. Curved needles are advanced through the struts 383 and into the patient tissue 53 (see needles 400 and their inwardly-curved tissue penetration path. When the needles 400 are fully advanced into a patient their tips may be coupled to the sutures 450, so that by retracting the needles the sutures are retracted and tensioned the prosthetic device 270 is held into firm abutting contact with the patient's internal tissue surface.

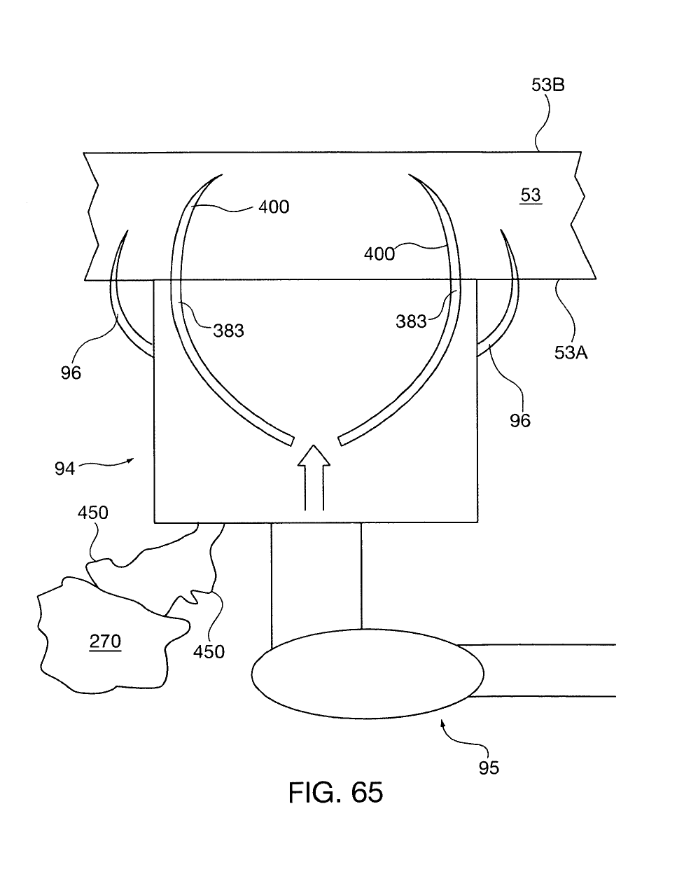

FIG. 65 shows schematically a proposed internally oriented fixation device 94 that may be inserted into a patient body cavity by way of a laparoscope 95. Struts 383 orient and advance needles 400 into a curved path from inside to outside the patient's body tissue 53. Hooks 96 anchor the fixation device into firm contact with the tissue's interior surface 53A. As the needles 400 are retracted from the patient, the mesh 270 is tensioned by any of the anchoring systems envisioned.

Although various embodiments which incorporate the teachings of the present invention have been shown and described in detail herein, those skilled in the art can readily devise many other varied embodiments that still incorporate these teachings.

What is claimed is:

1. A method for implanting a prosthetic device in a patient's tissue, comprising:
introducing in proximity to an implantation site in a patient's tissue a prosthetic device having a plurality of generally linear fixation devices coupled thereto;
advancing a fixation instrument having a distal tip along an advancement path in proximity to the implantation site;
establishing a plurality of fixation sites in the patient's tissue for implantation of the prosthetic device by deploying from the fixation instrument distal tip at least one leg corresponding to each fixation site, and abutting the leg against the patient tissue; and
orienting the prosthetic device into tensioned abutting contact with the patient tissue at each fixation site by maneuvering the generally linear fixation devices coupled thereto in cooperation with the corresponding leg.

2. A method for implanting a prosthetic device in a patient's tissue, comprising:
introducing in proximity to an implantation site in a patient's tissue a prosthetic device having a plurality of fixation devices coupled thereto;
advancing a fixation instrument having a distal tip along an advancement path in proximity to the implantation site;
establishing a plurality of fixation sites in the patient's tissue for implantation of the prosthetic device by deploying from the fixation instrument distal tip at least one leg corresponding to each fixation site, the respective leg deployment translating a retrograde path relative to the advancement path, and abutting the leg against the patient tissue; and
orienting the prosthetic device into tensioned abutting contact with the patient tissue at each fixation site by maneuvering the fixation devices coupled thereto in cooperation with the corresponding leg.

3. A method for operating a prosthetic implantation apparatus for implanting a prosthetic device in a patient's tissue, the apparatus having: a fixation instrument having a distal tip for advancement into patient tissue and a plurality of legs deployable from the distal tip along a respective deployment path, the legs capable of being coupled to generally linear fixation devices that are coupled to the prosthetic device, comprising:
introducing in proximity to an implantation site in a patient's tissue a prosthetic device having a plurality of generally linear fixation devices coupled thereto;
advancing the fixation instrument distal tip into the patient's tissue along an advancement path in proximity to an implantation site;
establishing a plurality of fixation sites in the patient's tissue for implantation of the prosthetic device by deploying from the distal tip at least one leg corresponding to each fixation site, and abutting the leg against the patient tissue;
coupling the legs to a respective prosthetic device linear fixation device, if not so previously coupled; and
orienting the prosthetic device into tensioned abutting contact with the patient tissue at each fixation site by maneuvering the generally linear fixation devices coupled thereto in cooperation with the corresponding leg.

4. A method for operating a prosthetic implantation apparatus for implanting a prosthetic device in a patient's tissue, the apparatus having: a fixation instrument having a distal tip for advancement into patient tissue and a plurality of legs deployable from the distal tip along a respective deployment path retrograde the intended advancement path, the legs capable of being coupled to fixation devices coupled to the prosthetic device, comprising:
introducing in proximity to an implantation site in a patient's tissue a prosthetic device having a plurality of fixation devices coupled thereto;
advancing the fixation instrument distal tip into the patient's tissue along an advancement path in proximity to an implantation site;
establishing a plurality of fixation sites in the patient's tissue for implantation of the prosthetic device by deploying from the distal tip at least one leg corresponding to each fixation site along a retrograde deployment path relative to the advancement path, and abutting the leg against the patient tissue; and
orienting the prosthetic device into tensioned abutting contact with the patient tissue at each fixation site by maneuvering the fixation devices coupled thereto in cooperation with the corresponding leg.

5. An implantation system for implanting a prosthetic device in a patient's tissue at an implantation site, the system comprising:
a prosthetic device having a plurality of generally linear fixation devices coupled thereto; and
a fixation instrument having: a distal tip for advancement into patient tissue along an advancement path terminating proximal the implantation site; a plurality of legs deployable from the distal tip along respective deployment, the legs having tips for abutment against patient tissue at a fixation site established thereby, the legs coupled to the prosthetic device generally linear fixation devices during their deployment, for orienting the prosthetic device into tensioned abutting contact with the patient tissue at each fixation site by maneuvering the generally linear fixation devices in cooperation with the corresponding leg.

6. An implantation system for implanting a prosthetic device in a patient's tissue at an implantation site, the system comprising:
a prosthetic device having a plurality of fixation devices coupled thereto; and
a fixation instrument having: a distal tip for advancement into patient tissue along an advancement path terminating proximal the implantation site; a plurality of legs deployable from the distal tip along respective deployment paths that are retrograde the intended advancement path, the legs having tips for abutment against patient tissue at a fixation site established thereby, the legs coupled to the prosthetic device fixation devices during their deployment, for orienting the prosthetic device into tensioned abutting contact with the patient tissue at each fixation site by maneuvering the fixation devices in cooperation with the corresponding leg.
